SEQUENCE LISTING (1 sequence)

SEQ ID NO: 1 - protein, 268 aa, Homo sapiens
MAVNVYSTSVTSDNLSRHDMLAWINESLQLNLTKIEQLCSGAAYCQFMDMLFPGSIALKKVKFQAKLEHEYIQNFKILQAGFKRMGVDKIIPVDKLVKGKFQDNFEFVQWFKKFFDANYDGKDYDPVAARQGQETAVAPSLVAPALNKPKKPLTSSSAAPQRPISTQRTAAAPKAGPGVVRKNPGVGNGDDEAAELMQQVNVLKLTVEDLEKERDFYFGKLRNIELICQENEGENDPVLQRIVDILYATDEGFVIPDEGGPQEEQEEY

(19) United States
(12) Patent Application Publication
(10) Pub. No.: US 2007/0212681 A1
(43) Pub. Date: Sep. 13, 2007

(54) Cell canaries for biochemical pathogen detection

(76) Inventors: Benjamin Shapiro, Washington, DC (US); Pamela Abshire, Silver Spring, MD (US); Elisabeth Smela, Silver Spring, MD (US); Denis Wirtz, Washington, DC (US)

Correspondence Address: DYKEMA GOSSETT PLLC, 10 S. WACKER DR., STE. 2300, CHICAGO, IL, 60606, US

(21) Appl. No.: 11/215,136

(22) Filed: Aug. 30, 2005

Related U.S. Application Data
(60) Provisional application No. 60/605,653, filed on Aug. 30, 2004.

(51) Int. Cl. C12Q 1/70; C12Q 1/68; C12M 3/00

(52) U.S. Cl. 435/5; 435/6; 435/287.2

(57) ABSTRACT

Methods and compositions for the reliable detection of pathogens are presented. The invention uses cells as novel pathogen detection agents, exploiting pathogen-specific pathways and apoptosis.

### CROSS-REFERENCE TO RELATED APPLICATION

This application claims priority to 60/605,653, filed Aug. 30, 2004, entitled CELL CANARY, the entirety of which is herein incorporated by reference.

### STATEMENT REGARDING FEDERALLY SPONSORED RESEARCH OR DEVELOPMENT

The subject matter of this application may in part have been funded by the National Science Foundation, ECS0225489, the United States Department of Defense, Md. Procurement H9823004C0470, and the United States Air Force, FA95500410449. The government may have certain rights in this invention

### INCORPORATION-BY-REFERENCE OF MATERIAL SUBMITTED ON A COMPACT DISC

Not Applicable.

### FIELD OF THE INVENTION

The invention relates to apparatus for pathogen detection, methods of detecting pathogens using the apparatus, and methods of making the apparatus.

### BACKGROUND OF THE INVENTION

Anthrax, plague, smallpox, Clostridium botulinum toxin, salmonella, Ebola virus, and Escherichia coli are just a few of the threats that can be spread in a bioterrorism attack, whether through a dirty bomb or through the food supply. In such cases, fast, accurate, precise, and sensitive detection are essential such that preventative actions can be taken or the most effective treatments supplied to the affected.

Despite a tremendous amount of research and development efforts, biochemical pathogen detection is plagued with false positive results—that is, the pathogen is detected when not present in a sample. In some assays, the rate of false positives is unacceptably high, rendering the test just barely more useful than no test at all. Table I summarizes some of the commercially available pathogen detection systems (Clark et al., 2001; Johnson-Winegar, 2000); Table 2 presents the main detection technologies, many of which are complex, space-consuming, time-consuming, and costly.

TABLE 1
Commercially available or in-development air-borne pathogen detection systems

| | Pathogen | Components, and | | |
|---|---|---|---|---|
| Device | Detection | their Methods of | | Comments on |
| Name | Capability | Operation | Sensitivity | Capabilities |
| Chemical | Detects, identifies | Ion mobility | Not available | 1 minute |
| Agent | and quantifies G- | spectrometer | (is proprietary | detection time |
| Monitor | and V-type nerve | | or restricted | |
| (CAM) | agents and H- | | information) | |
| | type blister agents | | | |
| Biological | Detects and | Triggers alarm by UV | 10 ACPLA* | 30 minute |
| Integrated | identifies 8 | particle sizer. | | detection time. |
| Detection | biological warfare | Identifies pathogen | | |
| System | agents | by chemical biological | | |
| (BIDS) | simultaneously | mass spectrometer | | |
| | | and antibody-based | | |
| | | biological detector | | |
| Interim | Detects biological | Collect sample by wet | 15 ACPLA* | 45 minutes |
| Biological | warfare agents, | wall cyclone. | | detection time; |
| Agent | used on ships. | Identifies pathogen | | large system: |
| Detector | | by immunochemical | | 7.5 ft³, 200 lbs. |
| (IBAD) | | assays | | |
| Joint | Detects and | Not available | 15 ACPLA* | 20 minutes |
| biological | identifies 10 | (proprietary or | | detection time; |
| point | biological warfare | restricted | | is under |
| detection | agents | information) | | development; |
| system | simultaneously | | | will replace |
| | | | | other biological |
| | | | | agent detectors |
| Joint | Detects, identifies | Collects sample by | Not available | Near real time |
| chemical | and quantifies | pulsed air sampler. | | detection; is |
| agent | nerve, blister and | Detection method is | | under |
| detector | blood agents | not available | | development. |
| | inside aircraft and | (restricted) | | |
| | ship interiors | | | |

*ACPLA, Agent Containing Particle per Liter of Air

TABLE 2
Currently available detection means

| Technology | Detection Method | Notes |
|---|---|---|
| Immuno-based | Antibody binds to antigen | Depends on interactions between |
| | | antibody and antigen, which is often |
| | | not sufficiently specific. |
| Polynucleotide | Polynucleotides on the sensor that | Detection depends on specific |
| probes | are complementary to specific | interaction between probe and |
| | pathogen polynucleotide sequences | target, while being able to |
| | bind to the target pathogen DNA | compensate for mutation without |
| | or mRNA. | sacrificing binding fidelity. |
| Gene chips | Detects DNA from pathogen - a | Sample is cleaned of human DNA, |
| | subset of polynucleotide probes | and DNA from known pathogens is |
| | | amplified by polymerase chain |
| | | reaction (PCR) and then detected by |
| | | binding to specific elements of the |
| | | gene chip array. Unknown |
| | | pathogens pose a challenge. |
| Ion mobility | Separates and detects electrically | Useful for chemical detection but |
| spectrometry | charged particles (ions) based on | unreliable for pathogen detection. |
| | how fast they travel through an | |
| | electrical field. | |
| Mass | Requires field spectroscope and | DNA is amplified by PCR, and |
| spectrometry | sample to be vaporized | fragments are separated based on |
| | | the ratio of their mass to electrical |
| | | charge (m/z). The relative number |
| | | of each of the four nucleotides is |
| | | characteristic of the pathogen. Also |
| | | useful for toxin detection. |
| Infrared | Chemical bonds within a molecule | Good for detecting chemical vapors |
| | have “resonant frequencies”, the | over long (5 km) distances. |
| | amount of energy that triggers a | |
| | characteristic motion for a | |
| | particular type of bond; this | |
| | motion can be detected using | |
| | infrared light. | |

Unacceptable rates of false positives result in part due to the complexity of biological systems, the complex interaction with pathogens, and the inability of current sensor systems to differentiate subtle distinctions between the many possible interactions. Even those based on molecules such as DNA, RNA, and antibodies are not always able to differentiate between agents that are harmful and similar agents that are benign. While current systems are valuable tools for detecting some pathogens, the costs, labor, complexity and most importantly, the rate of false results, mitigate their effectiveness. Furthermore, current systems take approximately 24 hours to determine the pathogen in a sample if there is no other information to narrow down the type. A faster system is needed to enable patients to get life-saving treatment as soon as possible.

### SUMMARY OF THE INVENTION

In a first aspect, the invention is directed to devices for detecting at least one pathogen. The device contains at least one cell that produces a signal upon contact with the pathogen, at least one cell clinic on a surface of a chip for containing the cell; and an on-chip means for detecting the signal. The presence of a pathogen correlates to the production of the signal by the cell. In some cases, more than one signal is detected; in other cases, one cell can detect multiple pathogens, generating distinct signals in response to each pathogen. The cells can be engineered to produce signals that can be detected; modifications can include the introduction of exogenous markers, polynucleotides, viruses, etc. Markers include polypeptides, nanoparticles, polypeptides and dyes. Examples of detectable signals include fluorescence, fluorescence resonance energy transfer, light emission or cessation of light emission; a change, such as a change in cell resistance, cell impedance, cell capacitance, cell ion concentration, cell or medium pH, carbon dioxide concentration, nutrient concentration, cell waste concentration, cellular mechanical properties, cell position, cell number, and temperature. Changes are detected, for example, by sensors. Examples of sensors include those for fluorescence, carbon dioxide, pH, ion concentration, cell resistance, cell impedance, cell capacitance, cell waste, cellular mechanical properties, cell position, cell number nutrient and temperature. Fluorescence sensors can include current mode pixels, APS pixes, and single-photon avalanche detectors. Sensors can also include optical filters, such as notch filters, band-pass filters, and low-pass optical filters. The optical filters can also include absorbing dyes, interference filters, distributed Bragg reflectors, and patterned light-blocking layers. The chip of the can include integrated circuitry, such as that from complementary metal oxide semiconductor technology. The cell clinics can also include actuated lids, such as those containing polypyrrole. The lids can also have semi-permeable membranes. Finally, the device can also contain light sources that direct light to cells in the clinic.

In a second aspect, the invention is directed to devices for detecting pathogens. The device has a plurality of cells that produce at least one signal upon contact with a pathogen, a cell clinic comprising a plurality of vials on a surface of a chip for containing the cells, each vial containing at least one cell responsive to a specific pathogen; and a means for detecting responses of the cells to the pathogens. The cells can specifically respond to multiple pathogens, generating specific signals in response to contact with each pathogen. In general, the device can be thought to functions, wherein n and m represent integers greater than or equal to 1, and m cells specifically respond to n stimuli, wherein an nth stimulus elicits a specific response in an mth cell. Pathogens that are detected can elicit sequences of signals from at least one cell over time; which can be detected. The device can contain an array of sensors. The plurality of vials can contain different types of cells.

A third aspect of the invention provides for methods of analyzing a sample. The method includes the sample into a cell canary, the cell canary having a cell that produces a specific signal in response to a pathogen; and assaying the cell for the signal, wherein detecting the signal correlates with the presence of the pathogen. The signal can be fluorescence.

In a fourth aspect, the invention provides methods for making a device for detecting a pathogen. The device is made by fabricating on a chip at least one means for detecting a signal from at least one cell having a specific signal in response to a pathogen; fabricating at least one cell clinic on a surface of the chip for containing the cell; and loading the cell into the cell clinic.

### BRIEF DESCRIPTION OF THE DRAWINGS

FIG. 1 shows an embodiment of an on-chip pathogen sensor.

FIG. 2 shows an embodiment of a cell clinic.

FIG. 3 shows an embodiment wherein fluorescent signals are detected by an on-chip contact imager.

FIG. 4 shows schematics of current mode (FIG. 4A) and voltage mode (FIG. 4B) pixels.

FIG. 5 shows a schematic diagram of row logic and readout chain for one pixel, (FIG. 5A) and timing of corresponding signals (FIG. 5B).

FIG. 6 shows an example of a CMOS capacitance sensor for cell proximity detection.

FIG. 7 shows a fully differential pixel structure for fluorescence detection and a timing diagram of its control signals.

FIG. 8 shows a graph of the variation of sensor voltages with electrode distance.

FIG. 9 shows a graph showing sensor distance resolution as a function of cell proximity.

FIG. 10 shows a plot of sensor responses to living cells and calibrated cell capacitances.

FIG. 11 shows a graph of sensor response to variations in cell viability.

### DETAILED DESCRIPTION

The invention provides compositions, devices, systems, and methods for the detection of pathogens, as well as their identification. The invention is based on the ability to use living cells to emit a detectable signal upon contact with a pathogen and the ability to detect cell responses on-chip. The invention takes advantage of the observation that cell responses to pathogens are more informative and definitive than conventional analytical methods and devices. It also takes advantage of the rapid response of cells to pathogens.

1. Advantages

The advantages of using the compositions, devices, systems, and methods of the invention include:
(1) reduced false positive readings;
(2) ability to use simply as a detector to detect presence of pathogens;
(3) ability to engineer the invention to be both a detector of a pathogen and/or an identifier that would determine the type of pathogen present;
(4) the ability to create built-in positive and negative controls, enhancing the validity of the invention's results;
(5) potentially increased speed in obtaining the results;
(6) potentially lower cost compared to conventional, less reliable detection systems; and
(7) increased confidence in the results.

These aspects greatly increases the ability of decision makers to take the most appropriate action in the case of, for example, a terrorist attack or in food contamination, sparing both unneeded expense and most importantly, lives.

2. Biological Basis for Invention

One key challenge in pathogen detection is achieving reliable operation. Mistakes come in two types: false positives (or false alarms, meaning that the sensor detects a pathogen that is not there) and false negatives (a pathogen is present and the system does not detect it). In order to increase reliable operation, one can harness the specificity of biological systems.

The key concept behind the invention is that when a cell is exposed to a pathogen or other stimulus, certain biochemical pathways are triggered. The pathways that are activated depend on the type of cell and on the particular stimulus. What this means is that certain signaling events are triggered, such as the following: (1) the binding sites in the membrane can cluster or otherwise re-arrange themselves spatially; (2) molecules within the cell can be phosphorylated or de-phosphorylated; (3) the cell can begin to produce certain proteins in a particular order; (4) the concentrations of ions, hydrogen, and other chemical moieties in and around the cell can change (e.g., carbon dioxide, ions of calcium, sodium, potassium, chloride, hydrogen, water, oxygen). In addition, the cell can undergo various changes, such as (i) an increase or decrease in metabolism (causing a change in the uptake of nutrients, the production of waste, a change in temperature); (ii) a change in mechanical properties (membrane stiffness, skeletal stiffness); (iii) a change in shape (spreading out v. round); (iv) a change in attachment to the substrate and/or other cells (v) a change in the ability to reproduce (cell division); (vi) a change in physical location on the surface or in relation to other cells; and (vii) a change in electrical properties, such as impedance, resistance, capacitance and inductance. The cell response is unique to the pathogen or target.

Since different pathogens affect different cells in different ways, by sensing a subset of the above list of effects, different pathogens can be differentiated. In particular, a single cell can react to two different pathogens in different ways. If the responses are of different types, then two different sensors are used to monitor this one cell. If the single cell produces one type of response, e.g., a change in temperature, but the timing of this response is different for the two pathogens, then even one sensor can differentiate between the two pathogens. If different types of cells respond to different pathogens in different ways, then multiple cell types can be used to identify the pathogen.

Not all cell responses mentioned above are readily detectable. Cells can be engineered to make some of their responses to a pathogen detectable (Cell engineering is discussed more below). For example, a protein that is produced in a cell in response to a pathogen can be labeled with an exogenous fluorescent marker which can be detected optically.

To realize the invention, the target must have an effect on a cell and the effect must be detectable. The invention encompasses simple pathogen detection (i.e., a particular pathogen is present), identification of known pathogens (i.e., determine which pathogen is present), and/or characterization of new pathogens (to which family it belongs).

3. Engineering

In order to monitor cell behavior, one or more cells that can respond to the pathogen need to be kept alive and responsive. In order to detect the response(s) of the cell(s), one or more sensor(s) are required. In general, the cells need to be kept in a location where they can be monitored by the sensors. The invention therefore includes these three components: (1) responsive cells, (2) sensors, and (3) “cell clinics” to house the cells over the sensors. It is also useful to have circuitry for processing and comparing signals from the cells; for example, it is useful to perform signal conditioning (i.e., amplification, reduction of noise), cell stimulation (such as to elicit certain responses upon command), and interfacing and communication with external systems (displays, computers).

4. Pathogen Detection

If there is a cell with a unique response to a pathogen, then simple pathogen detection can be achieved by monitoring the cell to detect the occurrence of that response. This will not generally be the case, however. Therefore, in order to achieve reliable pathogen detection, monitoring of multiple cell responses from multiple cell types is beneficial. Multiple cells of the same type increase confidence in the result.

In order to identify a pathogen whose effects on a cell are known, it is necessary to monitor different signals from one or more cells. It is beneficial to have multiple types of cells in order to provide enough different signals to confidently identify the pathogen.

Characterization of new pathogens is challenging. Pathogens are of different types, for examples bacteria, viruses, and toxins. Within those types there are families, such as influenza and HIV viruses. Pathogens that belong to the same families typically share some of the responses that they induce in cells. Therefore, by monitoring a variety of cell responses, pathogen families can be identified. The more responses that are monitored, the more precisely the lineage of the pathogen can be identified.

Since the invention is based on the incredibly selective response of living cells, the rate of false positives is markedly decreased. If an early response to a pathogen can be monitored, then the speed of pathogen detection is increased.

5. Cell Death

Often, the end result of a pathogen attack on a cell is cell death. By monitoring cell death, it is also possible to detect the presence of pathogens, although this approach is slower than that of detecting other changes, such as signaling pathways. Cells suffering acute injuries swell and burst, thus spilling their cytoplasmic and nucleoplasmic contents onto their neighbors. In the body, this messy cell death often results in damaging inflammatory responses. This process is aptly called “necrosis” (Alberts et al., 2002).

However, cells can suffer another fate, one much less messy and that leaves their neighbors undisturbed. This quiet way of going is known as “programmed cell death,” or “apoptosis” (Greek for “falling off,” such as a leaf from a tree). Apoptosis can be detected using both morphological and biochemical criteria. Kerr and Wyllie described the morphological phenomena: the cell shrinks; the cytoplasm and nuclei condense, the nucleus fragments, chromatin condenses, the plasma membrane “blebs,” organelles are retained mostly intact, vacuoles form, and the DNA fragments. (Kerr et al., 1972; Wyllie et al., 1980) In the body, the apoptotic cells are cleaned up by macrophages or neighboring cells (Alberts et al., 2002).

Biochemically, the process of apoptosis proceeds as follows. Procaspases, which are protein-cleaving enzymes, are activated to caspases, which cleave proteins as well as other procaspases, resulting in a proteolytic cascade. Some of the key targets are the nuclear lamins (which are cleaved, contributing to the breakdown of the cell nuclei), and DNAses (activating them to cleave DNA) (Alberts et al., 2002). Importantly, apoptosis is an “all-or-nothing” response. Once past a certain point, there is no turning back, and the cell is sentenced to die (Alberts et al, 2002).

In turn, the procaspases are regulated by intracellular proteins, such as those of the Bcl-2 family. Some of these proteins have activity-blocking functions, while others promote procaspase activation. Inhibitor of apoptosis (IAP) proteins also have inhibitor roles with procaspases, either by inactivating them by binding to them or to prevent their activation (Alberts et al., 2002).

Apoptosis can be activated in three main ways: (1) unavailability of survival signals, such as growth factors; (2) conflicting signals during the cell cycle; and (3) recognition of a specific molecule at the cell surface. Pathogens induce apoptosis via their virulence determinants, which interact with components of the apoptotic pathway or interfere with transcription of genes that promote cell survival. Pathogenic bacteria kill cells by many different mechanisms, including: (1) pore-forming toxins, which “drill” holes into the cell membrane, causing the cytoplasm to leak; (2) introduction of toxins that are enzymatically active in the host cytoplasm; (3) specialized effector proteins secreted by some bacteria (type-III secretory systems); (4) “super antigens” that target immune cells, and (5) “other” modulators of apoptosis, such as toxins produced by Clostridium difficile and Bordetella pertussis (Weinrauch and Zychlinsky, 1999).

It is possible to detect cell death by monitoring capacitance. Cells detach from the substrate and cell footprints shrink during apoptosis. This can be sensed using a capacitive measurement since the impedance between the cell and its substrate depends on the contact area.

Definitions

Deleterious effect means “having a harmful effect; injurious.” While cell death exemplifies a deleterious effect, death is the epitome of deleterious. Less severe injuries are also included in this definition, such as changes that adversely effect cellular physiology and integrity.

Exogenous means “arising from outside;” the antonym is “endogenous.”

Pathogen means any agent that causes any kind of deleterious effect in a cell or an organism. Target and agent are used interchangeably. A pathogen can be a toxin, a bacterium, a virus, or fragments thereof; polypeptides proteins, peptides, etc.), polynucleotides (e.g., DNA and RNA, natural and unnatural; single-stranded, double-stranded and multi-stranded), or combinations thereof.

Pixel means a picture element containing a photosensor and transistors for converting electromagnetic radiation to an electrical signal.

Quantum dot means a nano-scale crystalline structure, usually made from cadmium selenide, that absorbs light and then re-emits it a couple of nanoseconds later in a specific color. The size of a quantum dot varies within the 1-10×10⁻⁹ m range.

Wafer and substrate mean semiconductor-based material including silicon, doped and undoped semiconductors, epitaxial layers of silicon supported by a base semiconductor foundation, and other semiconductor structures. Furthermore, when reference is made to a “wafer” or “substrate” in the following description, previous process steps may have been utilized to form regions or junctions in or over the base semiconductor structure or foundation. In addition, the semiconductor need not be silicon-based, but could be based on silicon-germanium, germanium, or gallium arsenide, among others.

Practicing the Invention

First, exemplary devices are presented that house the biological aspects of the invention and are used to detect pathogens. Second, biological aspects of the invention are presented. Finally, examples are provided to illustrate the invention.

1. The Engineering System

The engineering system has three major components: (1) closeable vials (cell clinics); (2) stimulating and sensing components; and (3) interface circuitry. After a brief example of one embodiment of a cell clinic, these three components are discussed in detail.

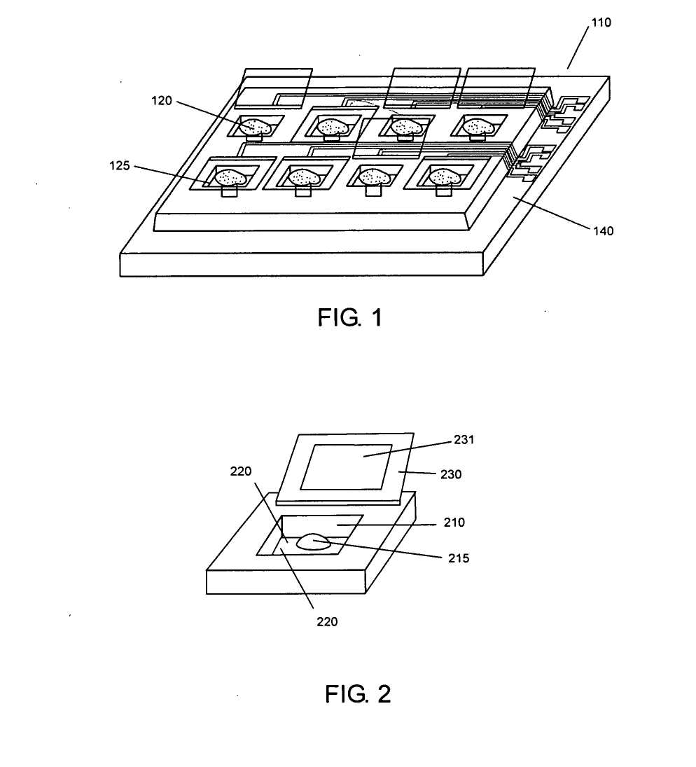

A pathogen sensing system contains one or more cell clinics 125 (FIG. 1) that contain and sustain a single cell 120 or a group of cells. Each cell clinic 125 is a meso-scale or micro-scale structure or device having a closeable cavity or vial on a substrate 140 in which at least one sensor has been defined through very large scale integration (VLSI) techniques.

FIG. 2 depicts one embodiment of a cell clinic 125. The cell clinic comprises a micro-scale vial 210 that houses at least one cell 215 and an actuated lid 230. The cell clinic 210 provides a controlled environment that sustains the life of the cell 215. The cells can be monitored by feedback control of their environment within each vial 210. Environmental variables that can be monitored can include CO₂, temperature, and pH.

In one embodiment, an actuated lid 230 covers the vial 210 to prevent the cells from leaving the confines of the vial and thus to ensure that the cells remain properly positioned over the sensors. The actuated lid 230 can include a semi-permeable membrane 231 that is used to allow molecules to pass through the lid while it is closed, such as nutrients, waste, and gases.

At the bottom of the vial are various sensors 220. Such sensors 220 measure the response of the cell to the pathogen. Such sensors 220 can be designed to measure cell optical activity, such as fluorescence, electrical activity, such as capacitance and resistance, chemical concentrations, such as ion concentration or pH, or cell metabolic activity, such as a change in pH.

Either at the bottom of the vial or elsewhere on the substrate surface can be located additional sensors 221 for monitoring the cells or their environment. In the case of adherent cells, electronic monitoring can be used as a measure of cell health. Chemical sensors can be used to measure the cell environment.

Various components of the cell canary are now described. The substrates that include the sensing components and circuitry are described first, then the cell clinics, and after that the sensors and other circuitry are described in more detail.

1. Chips

The substrates on which cell clinics can be built include chips with integrated circuits. The most commonly used technology for integrated circuitry today is complementary metal oxide semiconductor (CMOS) technology.

Because of the maturity of CMOS technologies, state of the art foundry processes can be used from many manufacturers, including AMI Semiconductor (Pocatello, Id.), Agilent Technologies, Inc. (Palo Alto, Calif.), Taiwan Semiconductor Manufacturing Company Ltd. (San Jose, Calif.), and Peregrine Semiconductor Corporation (San Diego, Calif.). Specific useful technologies include AMI 1.5 micron, AMI 0.5 micron, AMI 0.35 micron, TSMC 0.35 micron, TSMC 0.25 micron, TSMC 0.18 micron, Peregrine SOS 0.5 micron, Peregrine SOS 0.25 micron, and other technologies known to those skilled in the art.

2. Cell Clinics or Closeable Vials

The primary purpose of the cell clinics is to provide a place for the cells to live in a location that can be monitored by the sensor. Since the sensor is on-chip, the cell clinic needs to be physically over the chip surface. This can be achieved either by directly fabricating the cell clinics on the chip surface, or by fabricating them separately and joining them to the chip surface, either permanently or temporarily.

Preferably, the cell clinics provide a way to spatially separate cells or groups of cells (of the same or different types) so as not to confuse their signals, and/or to reduce cross-talk between cells above adjacent sensors and/or to ensure that the cells remain over the sensor. For example, if the signal is a change in chemical concentration, it is advantageous that the chemicals remain confined over the sensor, rather than diffusing over adjacent sensors. As another example, if the cells do not adhere the surface but stay suspended in solution, such as blood cells, then it is advantageous to physically hold the cells in place near the detector. The cell clinics may also serve other functions, such as to provide a more natural micro-environment for cell culture, which allows the cells to function more like they naturally do in the body.

In one embodiment of the invention, the cell clinics include vials that are positioned over the various sensors, so that the signals from the cells in that vial in response to pathogens can be clearly read by the sensor. Other sensors, such as those for chemical environment, can be positioned either inside and/or outside the vials.

The cell clinics may be semi-permeable. In other words, they may allow some things to reach the cells and not others. For example, they may allow food and waste and small toxins to pass through, but not viruses and bacteria.

The cell clinics are preferably fabricated directly upon the surface of the substrate containing the integrated sensors and circuitry. To do this, a process must be used that is compatible with the sensors and circuitry. This may place certain constraints upon the fabrication processes, such as a maximum temperature of approximately 350° C. (to avoid damaging the underlying circuitry) and the use of surface micromachining techniques (so that as much as possible of the chip surface can be covered with sensors and circuitry).

One material that can be used to form the vials is a thick film negative photoresist such as SU-8 (available from MicroChem Corporation; Newton, NA), which can be patterned photolithographically using standard mask aligners.

Alternatively, the cell clinic can be fabricated separately and then bonded to the surface. For example, it can consist of a poly dimethyl siloxane (PDMS) well fabricated by methods known to those in the art (such as micro-molding) and joined to the surface by a method known to those in the art (such as by treatment in an oxygen plasma to render the surface of the PDMS adhesive).

Depending on the type of sensor, there can be intervening layers between the cell clinics and the sensor. For example, an optical detector can be covered by transparent layers such as silicon dioxide, polymer, glass, etc. Other sensors may need to be in virtually direct contact with the cells or the cell medium (where direct contact does not necessarily preclude an intervening protein layer put down by the cell), such as ion sensors or electrodes.

Depending on the types of sensors, other processing may need to be done, either before or after fabrication of the vials, such as electroless plating or packaging.

In principal the vials can be any shape, such as rectangular, square, or round, but particular embodiments may require specific shapes. The dimensions of the cavities are adjusted according to cell size, number of cells in the vial, cell space and culture requirements (including gas exchange, nutrient flow, and waste discharge), and other variables, including the arrangement of the wells. For example, wells can be formed that are a few μm to several hundred μm square. The depth of the wells can range from 2 μm to 500 μm or more, more preferably the height should be appropriate for culturing a monolayer of cells, such as 10-100 μm. Larger overall dimensions are required if giant cells are used, such as oocytes (diameters of approximately 100 μm in mammals, 1000-2000 μm in frogs and fish).

One way to fabricate closeable vials is through the use of lids. Lids are used to hold the cells in the chamber over the sensors, and depending on the cell type and signal, to reduce cross-talk between adjacent vials (i.e., the reading of signals in one vial from cells in an adjacent vial), among other things. The vials can be opened and closed for cell loading, exposure to sample, or other purposes. To completely mechanically and/or chemically and/or electrically seal the lids, gaskets made of a film of a conforming material, such as a rubber-like polymer, can be situated around the perimeter of the vial opening. Other methods, such as chambers separated by hydraulically actuated membranes, can also be used to control the positions of the cells and their degree of isolation.

The vials can be opened and closed by electrically controlled lids. In one embodiment, the lids can be rotated by microfabricated bilayer actuator “hinges.” Such microactuators can be fabricated from conjugated polymers and noble metals. Conjugated polymers are characterized by alternating single and double bonds along the polymer backbone—a chemical structure that results in semiconductor-like properties. Conjugated polymers include polypyrrole, polyaniline, polythiophene, polyacetylene, etc. Other actuators are also possible, including other electroactive polymer actuators (such as ionic polymer-metal composites), thermal actuators, magnetic actuators, and others. Polypyrrole (PPy) actuators are preferred because they operate within a wide variety of aqueous salt solutions, including cell culture media (Jager et al., 2000). Lids can be fabricated from SU-8, BCB, polyimide, or other rigid structural materials.

To fabricate conjugated polymer Microsystems, standard microfabrication procedures can be used, including surface micromachining methods that involve sequential deposition and removal (etching) steps. Such procedures are known in the art (Skotheim et al., 1998; Smela, 1999).

The actuators are designed so that they can close the vial. In the case of bilayer hinges, the thicknesses of the layers and the length and width of the actuator are designed to achieve a rotation of 180° and a final height of the bottom of the lid to be at the top of the vial, as well as to achieve sufficient force to hold in the cells and to act against any other forces that must be overcome. The actuator design that is chosen is preferably the one that takes the least chip “real estate” to meet the requirements. Therefore, a large curvature (small radius of curvature) is preferable, and this is achieved by choosing an appropriate polymer to metal thickness ratio.

One conjugated polymer that can be used for the actuator is PPy doped with the large immobile anion dodecylbenzene sulfonate (DBS), PPy(DBS). Methods for fabricating and actuating such actuators have been given (Smela, 1999).

3. Stimulating and Sensing Components

The sensors for detecting the cell signals can be implemented using custom integrated circuits and fabricated on-chip using standard technology, such as CMOS. Cell signals can be detected using optical sensors, such as fluorescence sensors, luminescence sensors, and imagers, and electronic sensors designed to measure capacitance, resistance, impedance, or the small electrical signals generated by electrically active cells. Other sensing modalities are not excluded and are known to those skilled in the art.

The sensors for detecting the cell signals are fabricated on-chip, together with appropriate signal-processing circuitry. Means to enable the cell signals to be generated can also be integrated on the chip. For example, in the case of a fluorescence signal, the cell must be illuminated at an appropriate wavelength in order for the fluorescence to occur. In addition, instrumentation for loading cells into the cell clinics, monitoring cell behavior and health, and modulating cell behavior can be integrated/fabricated on the chip. Auxiliary circuits such as potentiostats to control the MEMS actuators and radio-frequency (RF) wireless interface circuits to provide communication links and power ultra-low power circuits can also be integrated onto the same substrate as the clinics.

a. In Situ Optical Sensing

(i) Fluorescence Sensors

One sensor that can be used to detect cell responses is a fluorescence sensor. Fluorescence is a brief light emission following the absorption of light. Molecules which can fluoresce are called fluorescent probes. Many fluorescent probes have been designed to localize components within a biological specimen or to respond to a specific stimulus. Because of the maturity of fluorescent probe technology, probes can be obtained from many manufacturers, including Invitrogen (Carlsbad, Calif.), Martek Biosciences Corporation (Columbia, Md.), and Sigma-Aldrich Corporation (St. Louis, Mo.). Specific useful probes can indicate a broad set of cellular features and properties such as ion concentration, proteins, nucleic acids, pH, membrane potential, and other features and properties known to those skilled in the art.

The two primary technical requirements for a fluorescence sensor are the ability to detect emitted fluorescent light of very low intensity at specific wavelengths and the ability to block light at other wavelengths which may interfere with the signal being detected. Fluorescence sensing systems typically have at least four components: (1) a light source; (2) optical filters; (3) detectors (i.e., light sensors); and (4) signal processing circuitry. The light source is designed to deliver sufficient optical power, the filters to be capable of discriminating wavelengths, and the detectors to distinguish fluorescent emission, even in the presence of interfering excitation light. The cell must be illuminated within an appropriate range of wavelengths in order for the fluorescence to occur. This “excitation” light can be generated a vertical-cavity surface-emitting laser (VCSEL) or a light emitting diode (LED) or by a semiconductor photon source. These can be separate components or integrated on-chip. The light can be directly shone on the cells or guided to the cells using an optical waveguide integrated on-chip.

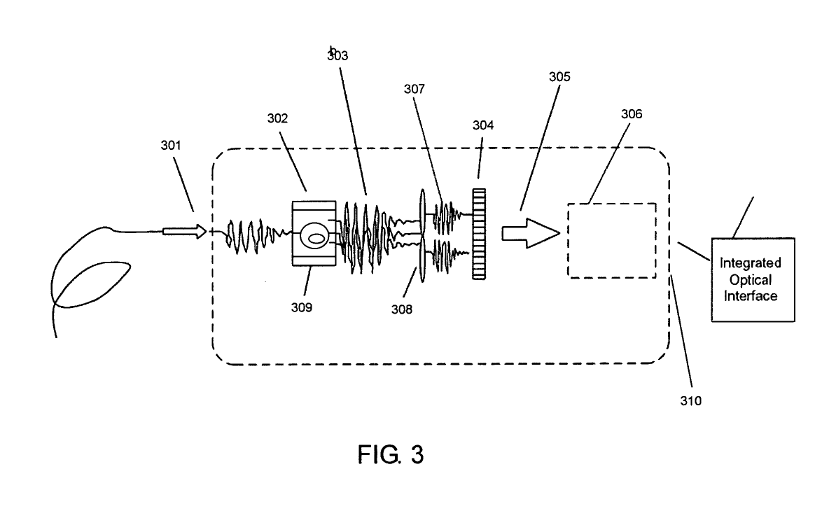

An example of an implementation that satisfies the technical requirements is a low noise integrated photodetector to detect the light signal, covered by an optical filter coating to block the interfering wavelengths. One embodiment is presented in FIG. 3. A cell 309 in a cell clinic 302 is exposed to a pre-chosen wavelength of light, compatible with the detectable signal (such as that which excites fluorescence in the target molecule or that which excites the FRET donor (see below)), emitted from a light source 301. An optical signal 303 is emitted that then passes through an emission light filter 308. The filtered optical signal 307 then strikes a photo sensor 304, which then transmits the signal electronically 305 and passes the signal to an integrated signal processor 306. 310 shows the integrated optical interface. The photo-sensor may be implemented according to known art, using an integrated circuit with low noise and low leakage current.

Many pixel designs are suitable for fluorescence sensors, including active pixel sensor (APS) pixels and single photon avalanche detector (SPAD) pixels. In the APS pixel, photocurrent is integrated onto the gate of a transistor. The integrated voltage is buffered by an amplifier in the pixel. Typically this amplifier is a unity-gain buffer implemented by a source follower, with the current source for the source follower common to the entire column. The integrated voltage is typically reset to the power supply voltage to start a new sampling period. In the SPAD pixel, a photodiode is biased at the edge of PN junction breakdown so that absorption of a single photon initiates an avalanche breakdown process. Additional circuitry monitors the pixel output to quench the avalanche events and measure their frequency of occurrence. APS pixels are easily implemented in standard CMOS technology, and SPAD pixels have been demonstrated in standard CMOS technology as well.

Optical filters can be notch or low-pass or band-pass that attenuate at the higher frequencies corresponding to the excitation wavelength, yet transmit at lower frequencies corresponding to the emission wavelength. Filters can be fabricated using microfabrication techniques.

In one embodiment, a stack of layers with alternating indices of refraction to filter the light can also be deposited. Alternatively, absorbing dyes can be incorporated into a polymer layer to attenuate undesirable wavelengths. Filters may also include a feature that physically blocks light from particular directions from reaching the detector (Roulet et al., 2001)

(ii) Luminescence Sensors

Another sensor that can be used to detect cell responses is a luminescence sensor. Luminescence refers to any light emission caused by an energy source other than heat, so fluorescence is a special case of luminescence in which the energy source is also light, and the light is emitted quickly following absorption. Many biochemical reactions produce luminescent light, which can be detected using a sensor similar to the fluorescence sensor. Luminescent light is also of very low intensity and can be detected using low noise photodetectors as previously described. Optical filtering is not mandatory for detection of luminescence, but use of optical filters can provide advantages such as improved signal quality and lower interference.

(iii) Image Sensors

Another sensor that can be used to detect cell responses is an imaging sensor. Conventional digital imaging technology can be used to acquire images of the cells. An imager having an array of high-resolution pixels can be used to detect cell positions, for placing cells in the clinic vials, and for preparing samples for presentation to cells. The imager can be used in either a normal imaging mode with optical elements such as lenses to focus the image onto an imaging array as in a standard camera or light microscope, or in a “contact” imaging configuration which does not use intervening optics and which generates a representation of a specimen directly coupled to the surface of the chip.

The photosensitive elements of the contact imager capture light that is transmitted through the cell, with a spatial resolution equal to the density of the photosensor array. Preferably, contact imagers are compatible with CMOS technology to enable the implementation of other sensors and circuitry on the same substrate (Culurciello and Andreou, 2004).

Many pixel designs are suitable for contact imagers, including current-mode pixels and active pixel sensor (APS) pixels. In the current-mode pixel, photocurrent serves as the input to a current mirror. The output is then switched to select the pixel of interest. The gate of the current mirror is driven by a current conveyor that clamps the voltage at which the photocurrent is measured. In the APS pixel, photocurrent is integrated onto the gate of a transistor. The integrated voltage is buffered by an amplifier in the pixel. Typically this amplifier is a unity-gain buffer implemented by a source follower with the current source for the source follower common to the entire column. The integrated voltage is typically reset to the power supply voltage to start a new sampling period.

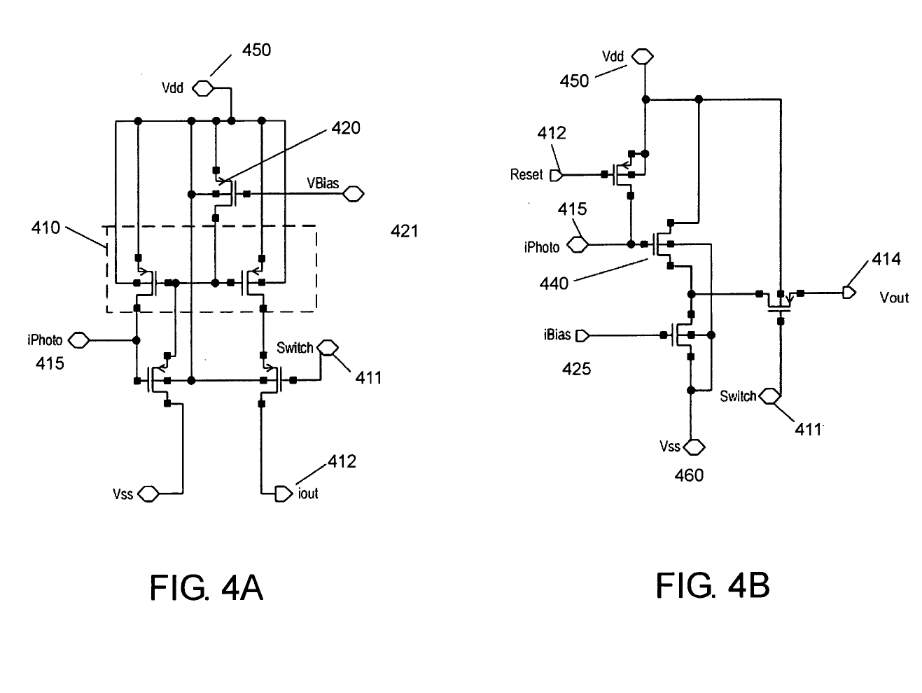

In one embodiment, FIGS. 4A and 4B are schematics of current mode and APS pixels respectively. In either case Vss 460 is a reference voltage. In the current mode pixel shown in FIG. 4A, photocurrent iPhoto 415 is input to a current mirror 410. The current mirror 410 acts as the collector load and provides a high effective collector load resistance, increasing the gain. The output of the current mirror 410 is selected using an nMOS switch 411 to select a pixel of interest. The gate of the current mirror 410 is driven by a current conveyor 420, which clamps the voltage at which the photocurrent iout 412 is measured. The current conveyer is biased by a MOS current source with gate voltage VBias 421. In the APS pixel shown in FIG. 4B, photocurrent iPhoto 415 is integrated onto the gate of transistor 440. The resulting voltage is reset using an nMOS switch 412 to the power supply voltage Vdd 450 to start a new sampling period. Transistor 440 is configured as a source follower and serves as a unity-gain amplifier that buffers the integrated voltage. The source follower is biased by a current determined by iBias 425. Although here it is shown as part of the pixel, the current source may be moved to the other side of the nMOS switch and be common for the entire column of pixels. The output of the pixel 410 is selected using an nMOS switch 411 to select a pixel of interest. Switch node 411 controls an nMOS switch to select the output signal Vout 414 from the pixel of interest.

The resolution of a contact imager is solely determined by its pixel size, in contrast with a conventional imager whose resolution is determined by the number of pixels in the array. Several techniques can be used in order to achieve a small pixel size. For example, all three MOS transistors can be N-type transistors. Photodiodes can be formed using n-type active area over the p-type substrate in order to avoid the large spacing requirements associated with the use of n-well regions. To reduce the number of contacts, there is preferably only one Vdd contact per pixel. The layout of the pixel array can be staggered so that one Vdd contact can be shared by the source follower input transistor of one pixel and the reset transistor of another. The top metal layer is used for routing the supply signal Vss and also serves to block light from all but the photodiode active area. In such a manner, a small pixel size with maximum optically active area is achieved.

To design a CMOS image sensor for individual cell detection, the effects a cell may have on the optical signal received by a sensor pixel is considered. Unlike in a natural scene, where the dynamic range of illumination may be greater than 100 dB, the illumination condition of an integrated biosensor system can be well controlled. For example, using a commercially available LED having an illumination power density of 50 mcd at 555 nm wavelength, a photon flux of 2.04×10⁶ photon/(um²·sec) is received by a pixel sensor placed approximately 10 mm away from the LED. Since most cells are nearly transparent, visibility can be enhanced by staining the cells using any appropriate stain for living cells, such as neutral red dye, which has an extinction coefficient (Eₑ) of 39000 cm⁻¹·M⁻¹. A dye concentration (C) of 0.1 M can be established in live cells. At such a concentration, the transmission rate (T) of illumination through a monolayer of cells 2 μm thick (I) can be calculated as:
=10^{-E}^{ₑ}^{×C×1}=10^{-39000×0.1×2×10}^{^{−4}}=0.166 (1)

Thus, 83.4% of the incoming light will be blocked. When the optical area of a pixel is comparable to or less than the cell size, an individual cell close to the pixel surface blocks a photon flux of 1.70×10⁶ photon/(μm²·sec). Assuming 40% quantum efficiency, a photodiode under a stained cell with a parasitic capacitance of 0.5 fF/μm² will generate a signal of 43 V/sec, which differs from the brighter background signal by 218 V/sec.

Pixels are integrated into arrays, and are associated with the interfacing circuitry; preferably, the arrays contain the smallest pixels and densest pixel space. Exemplary arrays include those having 8×8, 16×16, 24×24, 32×32, 64×64, 96×96, 128×128, and 256×256 pixel configurations, as well as larger arrays, and arrays that do not have square aspects in order to accommodate irregularly shaped regions of interest. In one embodiment, the interface circuitry is designed to scan all outputs continuously to monitor cell activity in every pixel during every cycle; alternatively, only a region of particular interest is scanned during every cycle. In yet other embodiments, a region of interest is selected and scanned in every cycle, while the entire array is scanned less frequently. Other options include incorporating asynchronous imaging techniques, such as time-based imaging or address event imaging (Culurciello et al., 2001) to send data only when an event of interest has occurred, such as when a detectable signal is emitted.

In one embodiment, the contact imager consists of a 96×96 active pixel sensor (APS) array, row and column scanners, column-wise readout circuits, and buffers and switches for input control and clock signals. Scanners and readout circuitry are implemented according to known art. The row and column scanner is implemented using a closed-loop shift register, and the output of the first stage of the row scanner serves as the clock signal for the column scanner.

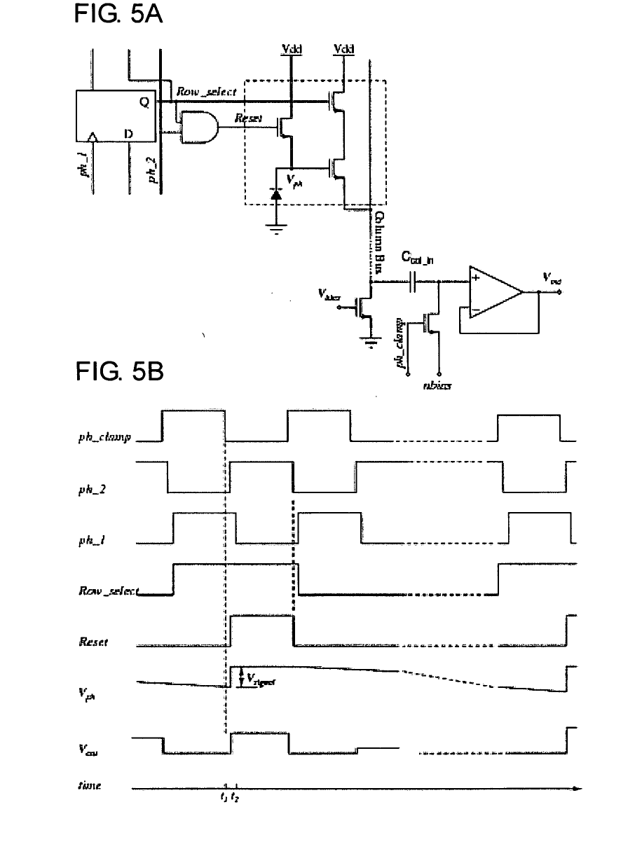

A schematic diagram of one embodiment of a pixel together with circuits for row logic and control, and a correlated double sampling (CDS) readout chain, is shown in FIG. 5A and a timing diagram (FIG. 5B). Three clocks are required to operate the imager: ph_1, ph_{—2, and ph}_clamp. They share the same frequency and should satisfy the phase relationships indicated by the dashed lines in FIG. 5B. The clock signal for the row scanner is ph_1. The output of one stage of the row scanner serves as the Row_select signal for all pixels in the corresponding row. The Reset signal initializes the integrated pixel value and is generated by performing a logic AND operation on the signals ph_2 and Row_select.

To suppress 1/f noise and fixed pattern noise (FPN) due to threshold variations of source-follower input transistors, column-wise correlated double sampling (CDS) can be performed (White et al., 1974). After the pixel is selected by Row_select and before Reset goes high, clock ph_clamp is high. At this point the integrated voltage signal is read out from the column amplifier. Clock ph_clamp then becomes low right before the positive edge of the Reset signal. This turns the input of the readout amplifier into a floating node capacitively coupled to the output of the selected pixel. After Reset goes high, the voltage is sampled again. To perform CDS properly, the three clock signals must satisfy the following phase shifts: clock phi is an inverted and slightly delayed copy of clock, and clock ph_clamp is an inverted and slightly advanced version of ph_2.

b. In Situ Electrical Sensing

Detection and processing of electrical signals generated by cells in response to stimuli are captured through electrodes that are close enough to the cells to detect their electrical response (action potentials)e. Preferably, at least one of the electrodes is within the cell clinic. These signals are then processed using CMOS circuits. Electrical measurements can be used to assay cell density (by measuring resistance) and cell health (by measuring capacitance). Cellular electrical activity is detected using voltage amplifiers with input signals that are provided from electrodes near the site of activity.

(i) Capacitance Sensors

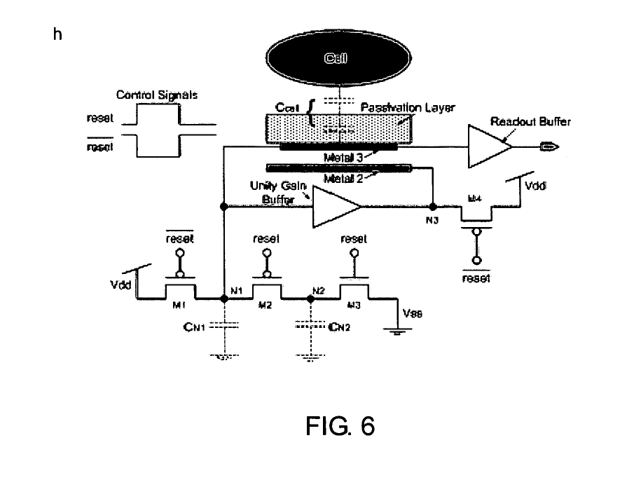

One sensor that can be used to detect cell responses is a capacitance sensor. An example of a CMOS capacitance sensor for cell proximity detection is shown in FIG. 6. In this example, the physical principle underlying operation of the sensor is charge sharing. The coupling capacitance C_{cell} is formed by the series combination of the capacitances between the cell and the passivation layer and between the passivation layer and the topmost metal electrode. C_{cell} varies inversely with the distance of the cell from the chip surface. The sensor circuit has two nodes N1 and N2 with parasitic capacitances CN1 and CN2. Charging and discharging of these nodes are controlled by a set of three MOSFET switches M1, M2 and M3, in two phases of operation. In the reset phase, switches M1 and M3 are turned on, charging N1 to Vdd and N2 to Vss, while switch M2 is off. The joint nodal voltage V_{N} as a result of the charge redistribution can be expressed as:

$${V}_{N} = \frac{( {C}_{N 1} + {C}_{cell} ) Vdd + {C}_{N 2} Vss}{{C}_{N 1} + {C}_{N 2} + {C}_{cell}} \qquad ( 2 )$$

where C_{cell} is the capacitance being sensed. As C_{cell} increases with increasing cell proximity to the surface, so does V_{N}. This determines the capacitance to voltage mapping. In order to maximize the sensitivity of the circuit, the parasitic nodal capacitances must be minimized. The sensor dynamic range also increases with increasing area of the metal electrode plate.

Continuing with FIG. 6, the topmost metal layer, (in this case metal3), forms the sensing electrode. The fringe capacitances between the metal3 plate and the substrate are shielded by means of a larger area metal2 plate below the sensing electrode. The large capacitance between metal2 and metal3 plates is cancelled by driving the metal2 shield with a potential that tracks the sensing electrode potential using a unity-gain buffer. The sensor in this example is designed for a supply voltage of +/−1.5 V and is fabricated in a commercially available 0.5 μm CMOS technology with three metal layers. Other examples of sensors include those having electrode areas of 20×20 μm², 30×30 μm² and 40×40 μm².

Continuing again with FIG. 6, in order to translate the sensor outputs to sensed capacitance values, the output voltages during the evaluation phase are subtracted from their corresponding reset voltages for offset cancellation. It follows from (2) that the sensed capacitance depends on this voltage difference according to the expression:

$$\begin{matrix}{C}_{cell} = \frac{( Vdd - Vss ) {C}_{N 2} - {V}_{diff} ( {C}_{N 1} + {C}_{N 2} )}{{V}_{diff}} \\ where & ( 3 ) \\ {V}_{diff} = {V}_{reset} - {V}_{eval} \\ and & ( 4 ) \\ {V}_{reset} = Vdd . & ( 5 )\end{matrix}$$

Here both Vᵣₑₛₑₜ and Vₑᵥₐₗ refer to the voltages before the readout buffer. The gain of the readout buffer must be considered in computing V_{diff} from the experimental readout values.

(ii) Electrical Amplifier

Another sensor that may be used to detect cell responses is an amplifier adapted to detecting the weak extracellular voltage signals generated by electrically active cells. In one embodiment, the amplifier circuit was an operational transconductance amplifier in a capacitive feedback configuration, designed for a midband gain of 100. A large feedback resistance implemented by a “pseudoresistor” pFET with gate connected to drain and bulk connected to source sets the low frequency cutoff, and the ratio of feedback capacitors sets the gain.

(iii) Electrodes

Also useful are electrodes within the cell clinics. Examples of sensing components include electrodes that can be used to measure impedance generated by cells growing in the cavities. Electrodes can also be used to stimulate electrically active cells. While aluminum is the most commonly used metal in commercial CMOS processes, it is often not compatible for bio-interfaces. Instead, maskless, electroless plating processes are used to provide a more suitable interface metal. The metal depends in part on the target cell characteristics; common metals include silver, gold, and platinum.

(iv) Resistance Sensors

Another sensor that can be used to detect a cell response is a sensor for cell resistance. This sensor comprises at least two electrodes and a means for determining the resistance between them. This can be accomplished by applying a small current and measuring the resulting voltage, or by applying a small voltage and measuring the resulting current. Such techniques are well known to those skilled in the art and use standard techniques of integrated circuit design.

(v) Impedance Sensors

Another sensor that may be used to detect a cell response is an impedance sensor that measures resistance and/or capacitance as a function of frequency. This sensor requires a means for sweeping the frequency and measuring the response. For example, the frequency can be varied using a circuit known as a voltage controlled oscillator. Such techniques are well known to those skilled in the art and use standard techniques of integrated circuit design.

c. Other Sensing Modalities

Other sensors can be used to detect the response of the cell and/or to monitor the health of the cell and/or monitor the cell medium. Based on the wide range of cell responses to a pathogen, a wide range of other sensors can be used. The following types of CMOS-based sensors, among others, are known to those skilled in the art: pH sensors, temperature sensors, ion sensors, oxygen sensors, carbon dioxide sensors, and NO sensors.

4. Integrated Circuitry for Signal Conditioning, Stimulating, Interfacing, and Communicating

Because interface circuitry can reduce the requirements for communicating sensitive analog values over long distances, encoders can be used to reduce the required communications to the minimum necessary for the required application. For example, data converters, such as analog-to-digital, replace an analog value susceptible to additive noise, with digital values that are restored at each subsequent stage of computation or communication. Radio-frequency (RF) wireless interface circuits can be integrated onto the same substrate as the clinics to provide communication links and to provide power to ultra-low power circuits.

2. Biological System

1. Cells Types and Culturing

Any cell type can be used, including prokaryotic and eukaryotic cells although mammalian cells are preferred, and human or human-derived cells are most preferred. Cells can be from other eukaryotic organisms, such as plants and fungi (including yeasts). Both primary culture cells and cell lines (available from the American Type Tissue Collection (ATCC); Manassus, Va.) are useful, although cell lines are preferred because of their immortality and ease of manipulation.

Useful cell types include pancreatic, intestinal, immune system, neuronal (including those of the brain, eye, nose and ear), lung, heart, blood, circulatory (lymph and blood), bone, cartilage, reproductive, glandular, enamel, adipose, skin, and hepatic.

Preferred cell types include Swiss 3T3 fibroblasts (e.g., ATCC Deposits CCL-163 and CCL-92 (Todaro and Green, 1963)). Mouse RAW (e.g., ATCC Deposit Nos. CRL-2278, TIB-50 and TIB-71) and human U937 (e.g., ATCC Deposit No. CRL-2367) cells are also preferred. Lung cells, more preferably, human lung cells; most preferably, lung cells that have the characteristic of being able to survive in air, are especially preferred. Table 3 presents some examples of human lung cell lines that can be modified for the methods and compositions of the invention.

TABLE 3
Examples of cell lines derived from human lung tissues

| Cell line name | ATCC deposit | Notes |
|---|---|---|
| LL 29 (AnHa) | CCL-134 | idiopathic pulmonary fibrosis |
| LL 47 (MaDo) | CCL-135 | |
| HEL 299 | CCL-137 | fetal |
| LL 24 | CCL-151 | |
| HFL1 | CCL-153 | fetal |
| MRC-5 | CCL-171 | |
| IMR-90 | CCL-186 | |
| LL 86 (LeSa) | CCL-190 | |
| LL 97A (AlMy) | CCL-191 | idiopathic pulmonary fibrosis |
| CCD-13Lu | CCL-200 | |
| CCD-8Lu | CCL-201 | |
| CCD-11Lu | CCL-202 | |
| CCD-16Lu | CCL-204 | |
| CCD 18Lu | CCL-205 | |
| CCD-19Lu | CCL-210 | |
| MRC-9 | CCL-212 | |
| CCD-25Lu | CCL-215 | |
| WI 38 | CCL-75 | |
| WI-38 VA-13 subline | CCL-75.1 | |
| 2RA | | |
| WI-26 VA4 | CCL-95.1 | |
| CCD-29Lu | CRL-1478 | emphysema |
| CCD-32Lu | CRL-1485 | |
| CCD-33Lu | CRL-1490 | |
| CCD-34Lu | CRL-1491 | |
| CCD-39Lu | CRL-1498 | hyaline membrane disease |
| HBE4-E6/E7 | CRL-2078 | bronchus |
| HBE4-E6/E7-C1 | CRL-2079 | bronchus |
| NL20 | CRL-2503 | bronchus; immortalized with |
| | | SV40 large T plasmid, p129 |
| NL20-TA | CRL-2504 | bronchus; immortalized with |
| | | SV40 large T plasmid, p129 |
| Hs 1.Lu | CRL-7000 | |
| Hs 115.Lu | CRL-7077 | bronchus |
| Hs 218.Lu | CRL-7180 | |
| Hs 389(A).Lu | CRL-7265 | |
| Hs 389(B).Lu | CRL-7266 | |
| Hs 394.Lu | CRL-7269 | |
| Hs 397.Lu | CRL-7272 | |
| Hs 401.Lu | CRL-7275 | |
| Hs 412.Lu | CRL-7285 | bronchus |
| Hs 417.Lu | CRL-7291 | bronchus |
| Hs 573.Lu | CRL-7344 | |
| Hs 888.Lu | CRL-7624 | |
| Hs 894(E).Lu | CRL-7635 | |
| Hs 907.Lu | CRL-7657 | |
| HE-LU (Rifkin) | CRL-7717 | fetal |
| Hs 468.Lu | CRL-7810 | |
| Hs 738.Lu | CRL-7868 | |
| BBM | CRL-9482 | bronchus; virus transformed |
| BZR | CRL-9483 | bronchus; virus transformed |
| BEAS-2B | CRL-9609 | bronchus; virus transformed |
| FHs 738Lu | HTB-157 | |

Suitable media and conditions for generating primary cultures are well known. The selection of the media and culture conditions vary depending on cell type and may be empirically determined. To keep cells dividing, serum, such as fetal calf serum (FCS) (also known as fetal bovine serum (FBS)), is added to the medium in relatively large quantities, 5%-30% by volume, depending on cell or tissue type. Other sera include newborn calf serum (NCS), bovine calf serum (BCS), adult bovine serum (ABS), horse serum (HS), human, chicken, goat, porcine, rabbit and sheep sera. Serum replacements may also be used, such as controlled process serum replacement-type (CPSR; 1 or 3) or bovine embryonic fluid. Specific purified growth factors or cocktails of multiple growth factors can also be added or sometimes substituted for serum. Specific factors or hormones that promote proliferation or cell survival can also be used.

Examples of suitable culture media include Iscove's Modified Dulbecco's Medium (IMDM), Dulbecco's Modified Eagle's Medium (DMEM), Minimal Essential Medium Eagle (MEM), Basal Medium Eagle (BME), Click's Medium, L-15 Medium Leibovitz, McCoy's 5A Medium, Glasgow Minimum Essential Medium (GMEM), NCTC 109 Medium, Williams'Medium E, RPMI-1640, and Medium 199. A medium specifically developed for a particular cell type/line or cell function, e.g., Madin-Darby Bovine Kidney Growth Medium, Madin-Darby Bovine Kidney Maintenance Medium, various hybridoma media, Endothelial Basal Medium, Fibroblast Basal Medium, Keratinocyte Basal Medium, and Melanocyte Basal Medium are also known. If desired, a protein-reduced or -free and/or serum-free medium and/or chemically defined, animal component-free medium may be used, e.g., CHO, Gene Therapy Medium or QBSF Serum-free Medium (Sigma Chemical Co.; St. Louis, Mo.), DMEM Nutrient Mixture F-12 Ham, MCDB (105, 110, 131, 151, 153, 201 and 302), NCTC 135, Ultra DOMA PF or HL-1 (both from Biowhittaker; Walkersville, Md.), can be used.

Media can be supplemented with a variety of growth factors, cytokines, serum, etc., depending on the cells being cultured. Examples of suitable growth factors include: basic fibroblast growth factor (bFGF), vascular endothelial growth factor (VEGF), epidermal growth factor (EGF), transforming growth factors (TGF and TGF.B), platelet derived growth factors (PDGFs), hepatocyte growth factor (HGF), insulin-like growth factor (IGF), insulin, erythropoietin (EPO), and colony stimulating factor (CSF). Examples of suitable hormone additives are estrogen, progesterone, testosterone or glucocorticoids, such as dexamethasone. Examples of cytokine medium additives are interferons, interleukins or tumor necrosis factor-α (TNF-α). Salt solutions may also be added to the media, including Alseverr's Solution, Dulbecco's Phosphate Buffered Saline (DPBS), Earle's Balanced Salt Solution, Gey's Balanced Salt Solution (GBSS), Hanks' Balanced Salt Solution (HBSS), Puck's Saline A, and Tyrode's Salt Solution. If necessary, additives and culture components in different culture conditions be can optimized as these can alter cell response, activity, lifetime, or other features affecting bioactivity.

In some instances, because of the confined space inside the cell clinics, a change in the amount of nutrients and ions may be desired; however, osmolarity should be maintained at tolerated levels. Defined media are often preferred to eliminate potential effects from undefined components, such as sera.

Most cells are adhesive and require a substrate to which they are able to attach. The surface on which the cells are grown can be coated with a variety of substrates that contribute to survival, growth and/or differentiation of the cells. These substrates include laminin, EHS-matrix, collagens, poly-L-lysine, poly-D-lysine, polyomithine and fibronectin. In some cases, three-dimensional cultures are desired, extracellular matrix gels can be used, such as collagen, EHS-matrix, or gelatin (denatured collagen). Cells can be grown on top of such matrices, or can be cast within the gels themselves.

If desired, the media can be further supplemented with reagents that limit acidosis of the cultures, such as buffer addition to the medium (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glyclclycine, N-2-hydroxyethylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminom-ethane (Tris), etc.). Frequent medium changes and changes in the supplied CO₂ (often approximately 5%) concentration can also be used to control acidosis. In some cases, because of the confined space inside the cell clinics, buffer concentrations can be adjusted to better control acidosis. For example, instead of 10 Mm or 25 Mm HEPES, 20 or 50 mM can be used.

Gases for culture typically are about 5% carbon dioxide and the remainder nitrogen, but optionally can contain varying mounts of nitric oxide (starting as low as 3 ppm), carbon monoxide and other gases, both inert and biologically active. Carbon dioxide concentrations typically range around 5%, but may vary between 2-10%. For many mammalian cells, carbon dioxide levels are usually kept in the range of 0.5% to 10%; more preferably 1% to 5%; and most preferably 2%±0.5%. However, carbon dioxide levels can be adjusted according to a cell's in vitro physiological requirements and empirically determined as necessary. Both nitric oxide and carbon monoxide, when necessary, are typically administered in very small amounts (i.e., in the parts-per-million (ppm) range), determined empirically or from the literature.

The temperature at which the cells grow optimally can be empirically determined, although the culture temperature usually is within the normal physiological range of the organism from which the cells are derived. In some cases, such as for storage of the cell-based sensor, cell growth and metabolism rates can be reduced by holding the cells at 0° C. to 4° C. until the sensor needs to be used, at which point they are returned to their physiologic temperature. Freeze-drying is another method for storage of cells.

2. Engineering of Cells for the Detection of Targets (Pathogens)

Any cell that responds to a pathogen can in principle be used in the cell canary. In some cases, it is useful to engineer the cells to give a particular type of response. Cells can be engineered to be simple detectors of targets, without specifically identifying them; they can also be engineered to detect and identify targets. Other embodiments provide both positive and negative controls, even within the same cell. To practice the invention, cells that respond to the target can be engineered so that the production of the proteins of interest, i.e., those that are indicative of the presence and/or identity of the target, can be detected.

Following the interaction of the cell with a pathogen, the cell will respond to it by activating a signaling pathway, which ultimately results in a change in cell function, for example, a change of cell motility or gene expression pattern, the onset of secretion of a chemo-attractant or growth factor, or the onset of cell death (necrosis or apoptosis), to name but a few examples. This signaling pathway is composed of a cascade of proteins and small molecules, including cell-membrane proteins (e.g., cell receptors), second messengers (e.g., calcium, phospholipids, etc.) and subcellular proteins (e.g., cytoplasmic and nuclear proteins), which sequentially interact with each other (“activate” each other). The optical detection of the activation of cell-membrane and subcellular proteins, e.g., their interactions with downstream effectors or upstream activators, can be accomplished by different methods, of which four are described here. These methods are only meant to be illustrative, and not limiting. For example, the cases below are based on the emission of light upon the binding of a “marker” with the protein. However, it is also possible to engineer the opposite response—an emission that is stopped (quenched) upon binding.

Method 1 consists in engineering the cell so that two proteins in the signaling pathway, which interact if the pathway is activated, are both tagged with exogenous fluorescent proteins (e.g., green or yellow or red fluorescent proteins) using conventional methods so as to preserve their biological function. When these two proteins begin to interact because the pathway has been activated, and the cell is being interrogated by fluorescent light, the fluorescent proteins are able to transfer energy to each other through a fluorescent resonant energy transfer (FRET). Through FRET microscopy, their interaction can be readily monitored. Here, the FRET “donor” is one of the two fluorescent protein-tagged proteins, and the “acceptor” is the other fluorescent protein-tagged protein. (The donor is the marker here). In other words, the cell is illuminated at one wavelength/frequency, and this light is absorbed by the donor and re-emitted by the donor at a different wavelength. The acceptor does not absorb this wavelength. When the donor and acceptor are physically close enough for FRET to occur, the light energy absorbed by the donor is transferred to the acceptor, and emitted by the acceptor at a third wavelength. The color change shows the binding event. Onset of FRET above background signal signifies significant interaction between these two proteins. The advantage of this approach is that it the fluorescent proteins are genetically encoded (in other words, the cell genome has been modified (engineered) to produce these proteins) and, therefore, do not have to be introduced by physical means into the cell through microinjection or bombing.

Method 2 consists in engineering the cell so that one of the two interacting proteins is tagged with a fluorescent protein (as done above) but the other protein is tagged with a quantum dot (QD). Like a fluorescent molecule, a quantum dot absorbs light of one wavelength and re-emits it at another. Using a quantum dot as the donor and the fluorescent-protein as the acceptor in a FRET detection scheme allows for continuous monitoring of protein-protein interaction without running into photo-bleaching problems that can occur when a fluorescent protein is continuously exposed to fluorescent light. (Photo-bleaching is a process whereby upon extended exposure to the excitation light, the fluorescent protein stops fluorescing.) The advantage of this approach is that the protein-tagged quantum dot can be monitored for long periods of times (hours and days). Moreover, the protein attached to the quantum dot does not have to be the full-length protein, but a functional fragment that can bind the other protein.

Organic and biomolecular fluorophores (Method 1) generally exhibit only moderate Stokes shifts between their excitation and emission spectra (in other words, the excitation and emission peak wavelengths are close together, making it difficult to filter the excitation wavelengths out while still allowing the emission wavelengths through to the detector), have relatively broad emission spectra (making the filtering even more challenging, since the peaks overlap, and reducing the number of fluorophores that can be used at once), and photo-bleach when monitored over extended periods of time (which causes the fluorescence to decrease as the fluorophore is illuminated at the excitation wavelengths). An exciting alternative to conventional fluorophores is quantum dots (QDs)(Doty et al., 2004). QDs offer the advantages of not photo-bleaching (unlike the green-fluorescent family of proteins), have narrow emission spectra, and have tunable excitations.

In one embodiment, the core of a QD consists of a semiconductor nanocrystal, such as CdSe, surrounded by a passivation shell, such as ZnS. Upon absorption of a photon, an electron-hole pair is generated, the recombination of which in ˜10-20 ns leads to the emission of a less-energetic photon. This energy, and therefore the wavelength, is dependent on the size of the core (smaller QDs have smaller wavelengths), which can be varied almost at will by controlled-synthesis conditions (Lidke and Arndt-Jovin, 2004). The surface is coated with a polymer that protects the QD from water and allows for chemical coupling to molecules.

The excitation spectra of QDs are a continuum, rising into the ultraviolet, and the emission spectra are narrow and slightly red-shifted to the band-gap absorption. Thus QDs with different emissions can be excited with a single excitation wavelength (Smith and Nie, 2004). The large extinction coefficient and the relatively high quantum yield of QDs, as well as their extraordinary photostability, permit the use of a low sample irradiance and prolonged imaging with a detection sensitivity extending down to the single-QD level.

QDs are commercially available (e.g., Quantum Dot Corp.; Hayward, Calif. and Evident Technologies; Troy, N.Y.) with a variety of conjugated or reactive surfaces, e.g., amino, carboxyl, streptavidin, protein A, biotin, and immunoglobulins. QDs are non-toxic to most cells. For example, tissue culture cells loaded with QDs survive for weeks without diminished growth or division, and the QDs persisted the entire time (Doty et al., 2004). In live animal studies, mice enjoyed healthy lives with QDs for months without obvious deleterious effects (Lidke and Arndt-Jovin, 2004). In the methods of the inventions, QDs coated with negatively charged dihydroxylipoic acid (DHLA), or with other hydrophilic coatings, are preferred.

QDs can be targeted to any specific area of the cell, or to any molecule in the cell. For example, to target the nucleus, QDs are coated with appropriate molecules, such as DNA-binding molecules (oligonucleotides, DNA-binding proteins, such as histones, transcription factors, polymerases and other molecules of the chromatin, DNA-binding dyes, or other small molecules, such as other base intercalators), or with protein oligomerization domains. QDs can be coated with specific receptor polypeptides.

Similarly, metallic nano-particles can be used to enhance any fluorescent signal, such as those made of gold and silver.

In one embodiment, QDs are coated with receptor polypeptides. In this state, when the receptor ligand site is empty, the QD is relaxed, and when excited, photoemits. However, in the presence of an analog-dye, there is no QD emission until the receptor ligand site is occupied. This is the result of fluorescence resonance energy transfer (FRET), which describes an energy transfer mechanism between two fluorescent molecules. A fluorescent donor is excited at its specific fluorescence excitation wavelength. By a long-range dipole-dipole coupling mechanism, this excited state is then non-radiatively transferred to a second molecule, the acceptor (analog-quencher dye). The donor returns to the electronic ground state. However, if the quencher is displaced from the proximity of the first fluorophore, it is then again free to fluoresce.

Nearby conducing metallic particles, colloids or surfaces can modify free-space spectral conditions of fluorophores such that the incident electric field “felt” by the fluorophore is increased (or decreased), and the rate of radiavity decay can also be modulated (Asian et al., 2004). The radiavity decay rate is that at which a fluorophore emits photons. Because the metallic nanoparticles need to be in close proximity to the fluorescent molecule (approximately about 5 nm), particles can be tagged with fluorescent molecules; or, in the case of polynucleotides (which have a low level of auto fluorescence at 260 nm and 280 nm), tagged with molecules that bind the polynucleotides, such as oligonucleotides, small molecules, or polynucleotide specific binding polypeptides. Similar approaches can be taken with proteins, but tagging with protein oligomerization domains, or those that interact with desired proteins in a specific manner (e.g., receptor-ligand, co-enzyme-enzyme, etc.).

QDs can be delivered to cells by any method known in the art. Biolistic projection and electroporation are two popular systems.

Method 3 consists in engineering the cell so that one of the two interacting proteins, or both, are tagged with a small-molecule fluorescent dye, as opposed to a fluorescent protein or a quantum dot. The advantage of this approach is that the tagging of the proteins can be accomplished using conventional biochemical methods of protein functionalization.

Method 4 consists in engineering a protein so that it carries a single fluorescent marker, such as a fluorescent protein. This protein is designed to specifically bind to the protein of interest. It has a conformation that does not fluoresce when illuminated. However, as a consequence of binding to the protein of interest, it changes conformation to one that is fluorescent when illuminated. When the protein of interest is produced by the cell, the marker protein will bind to it, and as a consequence the marker protein changes conformation and fluoresces under illumination. Thus when the protein is produced, a cell illuminated by the appropriate wavelength will fluoresce, which is detected by a fluorescence sensor. Alternatively, one could design a protein that changes conformation so that it goes from fluorescent to non-fluorescent upon activation.

The choice of target proteins (i.e., which pathways to label) is influenced by several variables, including: (1) if gene expression is turned on in the presence of a pathogen; (2) the association of proteins with each other upon activation of pathogen-specific pathways; (3) the quickness of the cell response and the point in the pathways that are detected.

a. Stains, Dyes, and Other Visual Labels

In most embodiments, the cells are engineered to fluoresce upon contact with a specific pathogen, or class of pathogens. Cell polypeptides whose expression is turned on or up-regulated are preferably fused by recombinant methods with fluorescent proteins (Table 4) such as the green fluorescent proteins. However, any detectable label can be used.

TABLE 4
Useful fluorescent protein partners

| Fluorescent Protein | Notes |
|---|---|
| β-glucuronidase (GUS) | Sensitive, broad linear range, non-isotopic. |
| Green fluorescent | Can be used in live cells; resists photo- |
| protein (GFP) and | bleaching |
| related molecules | |
| (RFP, BFP, YFP, etc.) | |
| Luciferase (firefly) | Polypeptide is unstable, difficult to |
| | reproduce, signal is brief |
| Secreted alkaline phosphatase | Chemo-luminescence assay is sensitive |
| (SEAP) | and broad linear range; some cells have |
| | endogenous alkaline phosphatase activity |

In Method 3, small molecule dyes can be used (see below) tethered to target proteins using conventional biochemical functionalization methods. This approach has the advantage of making use of robust dyes that can sustain continuous monitoring by fluorescence light, thus eliminating undesirable consequences, such as photo-bleaching. In some cases, classic labels can be used to detect a cell's response to a pathogen. The label can be coupled to a binding antibody, an interacting polypeptide, or to one or more particles, such as a nanoparticle. Suitable small molecule dye labels include fluorescent moieties, such as fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3 and 5; phycoerythrin; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives; pyrenes; pyridyloxazole derivatives; dapoxyl dyes; Cascade Blue and Yellow dyes; benzofuran isothiocyanates; sodium tetrafluorophenols; 4,4-difluoro-4-bora-3a,4a-dia-za-s-indacene. In some cases enzymatic moieties can be appropriate, such as alkaline phosphatase or horseradish peroxidase; and radioactive moieties, including ³⁵-[S] and ¹³⁵[I] labels. The choice of the label depends on the application, the desired resolution and the desired observation methods. For fluorescent labels, the fluorophore is excited with the appropriate wavelength, and the sample observed

Dyes and stains that are specific for DNA (or preferentially bind double stranded polynucleotides in contrast to single-stranded polynucleotides) can be used to exploit the phenomenon of fragmenting of DNA that occurs during apoptosis (Kerr et al., 1972; Wyllie et al., 1980). Such dyes include Hoechst 33342 (2′-[4-ethoxyphenyl]-5-[4-methyl-1-piperazinyl]-2,5′-bi-1H-benzimidazole) and Hoechst 33258 (2′-[4-ethoxyphenyl]-5-[4-methyl-1-piperazinyl]-2,5′-bi-1H-benzimidazole) and others of the Hoechst series; SYTO 40, SYTO 11, 12, 13, 14, 15, 16, 20, 21, 22, 23, 24, 25 (green); SYTO 17, 59 (red), DAPI, YOYO-1, propidium iodide, YO-PRO-3, TO-PRO-3, YOYO-3 and TOTO-3, SYTOX Green, SYTOX, methyl green, acridine homodimer, 7-aminoactinomycin D, 9-amino-6-chloro-2-methoxyactridine. Such stains and dyes can be loaded into the culture media. However, cell permeable stains and dyes are preferred. Tables 5 and 6 list many of the available polynucleotides-specific/chromosome specific stains currently available.

TABLE 5
Cell-permeant cyanine nucleic acid stains

| | | |
|---|---|---|
| Catalogue | | |
| #¹ | Dye Name | Ex/Em† |

Blue-fluorescent SYTO dyes

| | | |
|---|---|---|
| S11351 | SYTO 40 blue-fluorescent nucleic acid stain | 419/445 |
| S11352 | SYTO 41 blue-fluorescent nucleic acid stain | 426/455 |
| S11353 | SYTO 42 blue-fluorescent nucleic acid stain | 430/460 |
| S11354 | SYTO 43 blue-fluorescent nucleic acid stain | 437/464 |
| S11355 | SYTO 44 blue-fluorescent nucleic acid stain | 445/472 |
| S11356 | SYTO 45 blue-fluorescent nucleic acid stain | 452/484 |

Green-fluorescent SYTO Dyes

| | | |
|---|---|---|
| S34854 | SYTO 9 green-fluorescent nucleic acid stain | 483/503 |
| S32704 | SYTO 10 green-fluorescent nucleic acid stain | 484/505 |
| S34855 | SYTO BC green-fluorescent nucleic acid stain | 485/500 |
| S7575 | SYTO 13 green-fluorescent nucleic acid stain | 488/509 |
| S7578 | SYTO 16 green-fluorescent nucleic acid stain | 488/518 |
| S7559 | SYTO 24 green-fluorescent nucleic acid stain | 490/515 |
| S7556 | SYTO 21 green-fluorescent nucleic acid stain | 494/517 |
| S32706 | SYTO 27 green-fluorescent nucleic acid stain | 495/537 |
| S32705 | SYTO 26 green-fluorescent nucleic acid stain | 497/534 |
| S7558 | SYTO 23 green-fluorescent nucleic acid stain | 499/520 |
| S7574 | SYTO 12 green-fluorescent nucleic acid stain | 500/522 |
| S7573 | SYTO 11 green-fluorescent nucleic acid stain | 508/527 |
| S7555 | SYTO 20 green-fluorescent nucleic acid stain | 512/530 |
| S7557 | SYTO 22 green-fluorescent nucleic acid stain | 515/535 |
| S7577 | SYTO 15 green-fluorescent nucleic acid stain | 516/546 |
| S7576 | SYTO 14 green-fluorescent nucleic acid stain | 517/549 |
| S7560 | SYTO 25 green-fluorescent nucleic acid stain | 521/556 |

Orange-fluorescent SYTO dyes

| | | |
|---|---|---|
| S32707 | SYTO 86 orange-fluorescent nucleic acid stain | 528/556 |
| S11362 | SYTO 81 orange-fluorescent nucleic acid stain | 530/544 |
| S11361 | SYTO 80 orange-fluorescent nucleic acid stain | 531/545 |
| S11363 | SYTO 82 orange-fluorescent nucleic acid stain | 541/560 |
| S11364 | SYTO 83 orange-fluorescent nucleic acid stain | 543/559 |
| S11365 | SYTO 84 orange-fluorescent nucleic acid stain | 567/582 |
| S11366 | SYTO 85 orange-fluorescent nucleic acid stain | 567/583 |

Red-fluorescent SYTO dyes

| | | |
|---|---|---|
| S11346 | SYTO 64 red-fluorescent nucleic acid stain | 598/620 |
| S11343 | SYTO 61 red-fluorescent nucleic acid stain | 620/647 |
| S7579 | SYTO 17 red-fluorescent nucleic acid stain | 621/634 |
| S11341 | SYTO 59 red-fluorescent nucleic acid stain | 622/645 |
| S11344 | SYTO 62 red-fluorescent nucleic acid stain | 649/680 |
| S11342 | SYTO 60 red-fluorescent nucleic acid stain | 652/678 |
| S11345 | SYTO 63 red-fluorescent nucleic acid stain | 654/675 |

¹According to (Haugland, 2002), catalogue numbers are specific to Molecular Probes, Inc.
†Wavelengths of excitation (Ex) and emission (Em) maxima, in nm.

TABLE 6
Properties of classic nucleic acid stains

| | | | Fluorescence | |
|---|---|---|---|---|
| Catalogue #¹ | Dye Name | Ex/Em* | Emission Color | Applications† |
| A666 | Acridine homodimer | 431/498 | Green | Impermeant |
| | | | | AT-selective |
| | | | | High-affinity DNA binding |
| A1310 | 7-AAD (7-amino- | 546/647 | Red | Weakly permeant |
| | actinomycin D) | | | GC-selective |
| | | | | Flow cytometry |
| | | | | Chromosome banding |
| A1324 | ACMA | 419/483 | Blue | AT-selective |
| | | | | Alternative to quinacrine for |
| | | | | chromosome Q banding |
| D1306, D3571, | DAPI | 358/461 | Blue | Semi-permeant |
| D21490 | | | | AT-selective |
| | | | | Cell-cycle studies |
| | | | | Chromosome and nuclei |
| | | | | counterstain |
| | | | | Chromosome banding |
| D1168, D11347, | Dihydroethidium | 518/605 | Red [symbol] | Permeant |
| D23107 | | | | Blue fluorescent until oxidized to |
| | | | | ethidium |
| E1305, E3565‡ | Ethidium bromide | 518/605 | Red | Impermeant |
| | | | | dsDNA intercalator |
| | | | | Dead-cell stain |
| | | | | Chromosome counterstain |
| | | | | Flow cytometry |
| | | | | Argon-ion laser excitable |
| E1169 | Ethidium homodimer-1 | 528/617 | Red | Impermeant |
| | (EthD-1) | | | High-affinity DNA labeling |
| | | | | Dead-cell stain |
| | | | | Argon-ion and green He-Ne laser |
| | | | | excitable |
| E3599 | Ethidium homodimer-2 | 535/624 | Red | Impermeant |
| | (EthD-2) | | | Very high-affinity DNA labeling |
| | | | | Electrophoresis prestain |
| E1374 | Ethidium monoazide | 464/625 | Red | Impermeant |
| | | (unbound)** | | Photocrosslinkable |
| H1398, H3569‡ | Hoechst 33258 (bis- | 352/461 | Blue | Permeant |
| H21491 | benzimide) | | | AT-selective |
| | | | | Minor groove-binding |
| | | | | dsDNA-selective binding |
| | | | | Chromosome and nuclear |
| | | | | counterstain |
| H1399, H3570‡ | Hoechst 33342 | 350/461 | Blue | Permeant |
| H21492 | | | | AT-selective |
| | | | | Minor groove-binding |
| | | | | dsDNA-selective binding |
| | | | | Chromosome and nuclear |
| | | | | counterstain |
| H21486 | Hoechst 34580 | 392/498 | Blue | Permeant |
| | | | | AT-selective |
| | | | | Minor groove-binding |
| | | | | dsDNA-selective binding |
| | | | | Chromosome and nuclear |
| | | | | counterstain |
| H22845 | Hydroxystilbamidine | 385/emission varies | Varies | AT-selective |
| | | with nucleic acid | | Spectra dependent on secondary |
| | | | | structure and sequence |
| | | | | RNA/DNA discrimination |
| L7595 | LDS 751 | 543/712 (DNA) | Red/infrared | Permeant |
| | | 590/607 (RNA) | | High Stokes shift |
| | | | | Long-wavelength spectra |
| | | | | Flow cytometry |
| N21485 | Nuclear yellow | 355/495 | Yellow | Impermeant |
| | | | | Nuclear counterstain |
| P1304MP, | Propidium iodide (PI) | 530/625 | Red | Impermeant |
| P3566‡, P21493 | | | | Dead-cell stain |
| | | | | Chromosome and nuclear |
| | | | | counterstain |

¹According to (Haugland, 2002), catalogue numbers are specific to Molecular Probes, Inc.
*Excitation (Ex) and emission (Em) maxima in nm.
†Indication of dyes as “permeant” or “impermeant” are for the most common applications; permeability to cell membranes may vary considerably with the cell type, dye concentrations and other staining conditions. [symbol]
After oxidation to ethidium.
**Prior to photolysis; after photolysis the spectra of the dye/DNA complexes are similar to those of ethidium bromide-DNA complexes.

3. Reducing False Positives (False Alarms) and False Negatives (No Alarm in Presence of Pathogen)

Methods that can be used independently or together, can refine the identification of pathogens and reduce the occurrence of false positives. In a efirst method, cells are engineered to provide highly-redundant information; in a second method, cells engineered to detect different protein-protein interactions and placed in different wells are interrogated simultaneously and continuously.

Method A (for decreasing false positives). Here, more than one pair-wise interaction between proteins or protein and small molecules are monitored. Indeed, cells that are activated by similar, but different, pathogens often share proteins in their signaling pathways, which may render the identification of the pathogen difficult. Nevertheless signaling pathways often differ by (1) the kinetics (i.e., the rate and timing) of activation (and de-activation) of the proteins in the pathways, (2) the extent of activation of the proteins in the pathways. By monitoring the activation of several protein-protein interactions as a function of time, the invention can differentiate two pathogens and also differentiate an actual pathogen from a trivial change in media conditions. By tracking a sufficiently large number of protein pairs, the identification of the signaling pathway, and therefore the pathogen, is facilitated.

Method B (for decreasing false positives). In the second and complementary method, cells are engineered to detect only one type of protein-protein interaction or protein-small molecule interaction. But by placing differently engineered cells in different cell clinics, and exposing the cells simultaneously to the specimen containing (or not) the pathogen, we can effectively monitor several protein-protein interactions along the pathways at the same time. By tracking a sufficiently large number of protein pairs in different cell colonies at the same time, the identification of the signaling pathway, and therefore the pathogen, is facilitated.

To refine the identification of pathogens and further reduce the occurrence of false positives, the detection of protein-protein interactions can be complemented by the detection of variations in concentrations of small molecules. Indeed, the activation of a signaling pathway following the interaction of the cell with a pathogen will also typically affect the concentration of small molecules. Such small molecules include c-AMP, calcium and other ions, PIP2 and other phospholipids, to name but a few molecules. To monitor changes in the concentrations of these molecules one may use optical methods, such as conventional calcium indicators (which are fluorescent dyes).

Another complementary method to reduce the occurrence of false positives is best illustrated by the following example. To detect Clostridium difficile Toxin A, three cell clinics (instead of one) can be loaded with the same type of cells, all of which have been engineered to specifically detect this toxin. If all three vials indicate the presence of this toxin, then the user can conclude with higher certainty that the toxin is present.

Alternatively, different cell types can be engineered to respond to the same pathogen and be included in the device. Redundancy in detection is thus improved.

In addition, multiple proteins can be marked, either with QDs or a fluorescent protein or a fluorescent dye, and their signals monitored over time. Monitoring several such proteins in cells improves validity and accuracy. The proteins are chosen by their variation in activity over time in response to specific pathogens and non-pathogens.

Other controls will be apparent to one of skill in the art.

While the present invention is susceptible of embodiment in many different forms, there are shown in the drawings and described herein in detail specific embodiments thereof, with the understanding that the present disclosure is to be considered as an exemplification of the principles of the invention and is not intended to limit the invention to the specific embodiments illustrated.

### EXAMPLES

The following example is for illustrative purposes only and should not be interpreted as limitations of the claimed invention. There are a variety of alternative techniques and procedures available to those of skill in the art which would similarly permit one to successfully perform the intended invention.

### Example 1 Integrating MEMS Structures and CMOS Circuits for Bioelectronic Interface with Single Cells (an Example of a Cell Clinic)

Microvials 100 μm×100 μm and 10 μm to 20 μm high were made of SU-8 negative photoresist. The microvials were closed by SU-8/gold lids that were positioned by bilayer polymer actuators of PPy and gold. The clinics were fabricated on silicon wafers with electrodes leading to the hinges and to the interior of the vials. These structures have also been fabricated on top of custom VLSI circuitry designed to record signals from the cells within individual vials. All fabrication steps are performed at low temperature and are compatible with post-processing of the fabricated silicon die.

Because cells can escape from even deep microvials, a lid is included that can be closed after loading the cells into the vials. PPy doped with dodecylbenezenesulfonate (PPy(DBS)) is deposited over a layer of gold, which acts as the electrode through which potentials are applied as well as the constant volume layer of the bilayer, causing the bilayer to bend when the PPy changes volume. The PPy is electrochemically actuated: reducing the polymer pulls cations and water into the PPy(DBS), increasing the volume of the film, whereas oxidizing it expels the ions, decreasing the volume.

The first step in the fabrication process was to deposit and pattern a chromium layer onto an oxidized silicon wafer. Cr serves as an adhesion layer between Au and the substrate. Patterning it leaves openings for three-dimensional structures to be defined using the release method of differential adhesion. The next step was to evaporate a gold structural layer, which defines the electrodes, hinges, and lids. The structural layer was in some cases covered by a thin electroplated gold layer that roughens the surface to provide good mechanical interlocking between the PPy and gold. PPy(DBS) was electropolymerized on the hinge areas using a photoresist template; the PPy is deposited where the resist is absent. An SU-8 layer was then patterned to create lids, vials, and insulation for the wires. The gold layer was then etched in the final step to release the hinges. The hinges were attached to the substrate over those areas covered by Cr, but they were free over the areas with bare oxide since Au does not stick to silicon or silicon dioxide.

Some microvials were fabricated on top of custom VLSI circuitry. An array of ten bioamplifiers was fabricated in a commercially available 0.5 μm, 3-metal, 2-poly CMOS process, with each input taken differentially between electrodes defined in the VLSI layout and a common ground. The electrodes were “probe pads,” or openings in the top passivation layer that allow direct access to the metal layers. Electrodes were fabricated in two sizes, 25 μm×25 μm and 50 μm×50 μm.

The circuit was an operational transconductance amplifier in a capacitive feedback configuration, designed for a midband gain of 100 with supply voltages of +/−1.5 V. A large feedback resistance implemented by a “pseudoresistor” pFET with gate connected to drain and bulk connected to source sets the low frequency cutoff, and the ratio of feedback capacitors sets the gain.

The electrodes of the bioamplifier were fabricated using aluminum pads available in standard CMOS fabrication. The open A1 electrodes of the bioamplifier were covered with electrolessly-plated gold. The electroless plating process created a rough layer with a higher surface area. Electroless plating is preferred for this purpose because electroplating requires an electrical connection to the plated surface that will reduce sensitivity and increase noise during measurement. The plating baths were obtained from Technic, Inc. (Cranston, R.I.).

The bond wires were encapsulated to prevent shorting between them when an aqueous medium is placed on the chip and to isolate the CMOS packaging materials from the living cells. A variety of encapsulation materials have been used successfully, including room temperature vulcanite (RTV), silicone, and photopatternable polymers. For the RTV material, an opening in the center of the die was made using either a solid PDMS block, which was removed afterwards, or a hollow plastic pipette tip, which became part of the package. Silicone was also used in conjunction with a mold and cured to form an encapsulation barrier. Photopatternable polymers were also patterned using a simple mask and brief exposure to UV light to form an encapsulation barrier. A larger well was constructed using encapsulation materials and a section of plastic tube to hold the cell medium.

Biocompatibility of the materials was tested with bovine aortic smooth muscle cells (SMCs). SMCs stained with neutral red and cultured overnight adhered and formed cellular processes on the bottom of a fabricated vial (a silicon dioxide surface), on suitable encapsulation materials, and also on surrounding structures made of SU-8 photoresist and gold.

To test the bioamplifiers, SMCs were used because they are electrically active. Primary SMCs (Cell Applications, Inc.; San Diego, Calif.) were used. The test fixture was disinfected with 70% ethanol and then rinsed in sterile water. Cells were plated onto the fixture in sterile growth medium and allowed to adhere to the substrate for at least 12 hours in a humidified incubator at 37° C. in 5% CO₂. Testing was performed in Hank's balanced salt solution (HBSS). A ground electrode was provided by a gold wire placed in the extracellular medium.

Extracellular signals from the cells were in the μV to mV range. The bioamplifier amplified the signals from the cells and directly drove an off-chip buffer amplifier configured for unity gain. The buffered signal was monitored using an oscilloscope, and data were acquired using a GPIB interface. The size and placement of the cell relative to the recording electrode was a significant factor; the electrode should be smaller than each individual cell in order to obtain proper sealing (15-20 μm).

### Example 2 CMOS Fluorescence Sensor Design and Results

Fluorescence detection is a mature technology commonly found in microscopy and spectroscopy systems and widely used in biology labs worldwide. Such systems are typically large and require laboratory infrastructure for operation. In this work, the fluorescence sensor has been miniaturized for integration into the cell clinics vials for monitoring cells in real-time.

1. Design

For the fluorescence sensor, the primary design constraints were sensitivity and spectral selectivity. Fluorescence from the specimen in each cell clinic was expected to be weak (normally 10⁻⁴˜10⁻⁸ fc (footcandle), and 10⁻⁶ fc corresponds to 4.6 photons/100 μm²/s) (Herman, 1998). Though collection efficiency for the fluorescence signal was expected to be somewhat more efficient than in normal fluorescence microscopes due to the lack of intermediary optics, the extreme weakness of fluorescence still posed a substantial challenge in the development of an integrated optical sensor.

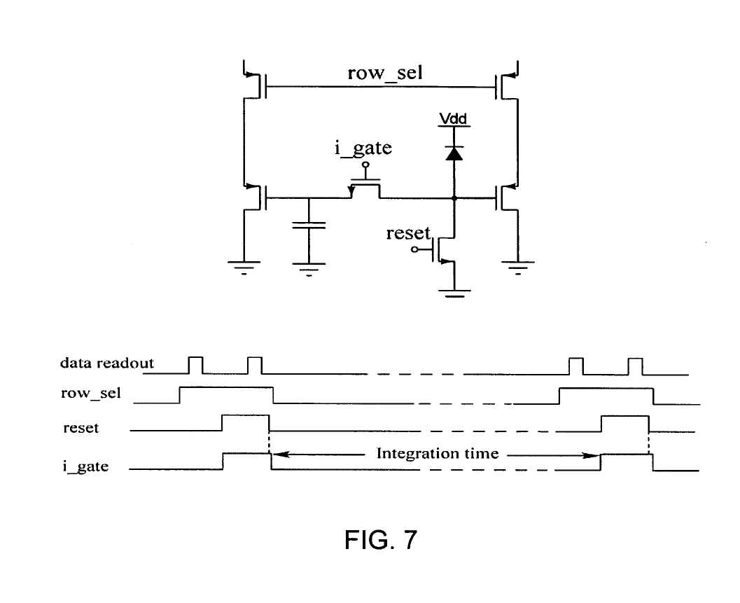

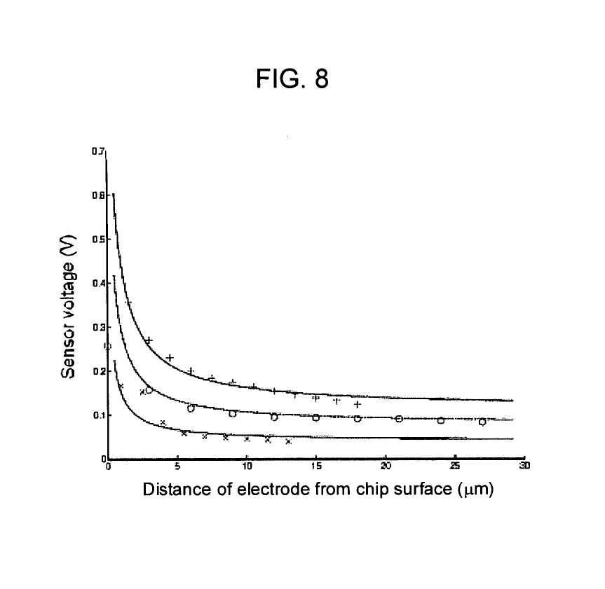

Conventional correlated double sampling (CDS) samples the photovoltage at the end of an integration period, then resets the integration node and samples the reset voltage. CDS can cancel deterministic offsets such as threshold voltage variations between transistor in different pixels, but does not reduce reset noise since the two samples compare reset values during two successive integration periods. In order to develop a pixel with low reset noise suitable for detection of weak fluorescence signals, a new pixel was designed using fully differential readout between samples acquired during a single integration period. The schematic of the pixel circuit is shown in FIG. 7. The pixel circuit consisted of six transistors and one capacitor. Two source follower input transistors were included in the pixel circuit and both were p-type transistors. P-type transistors were also adopted for row selection switches. To increase well capacity for the photodiode, an n-type transistor was used as reset transistor. An N-type MOSFET was also used for the isolation gate between the MOS capacitor and photodiode. Thus steady state could be reached during reset. Given a negligible leakage current of the capacitor, channel random noise of the isolation gate could be ignored while on, and possible offset due to threshold mismatch was also eliminated. The photodiode was implemented using an p⁺_nwell junction with nwell connected to the power supply. The capacitor could be implemented by a p⁺-nwell diffusion inside the same nwell as for photodiode. The pixel circuit needed three control signals instead of the usual two. These three signals were called reset, row_sel (row select), and i_gate (isolation gate). The operation was as follows: during the reset period, both reset transistor and isolation gate were “on” since they were n-type transistors, and the voltage on photodiode was very close to ground. The time required for the pixel to reach steady state was very short. Both the photodiode and the p⁺ diffusion shared the same reset noise since no current flowed through the isolation gate at the end of reset. The isolation gate was turned off immediately after the reset transistor was turned off. Therefore, the reset noise voltage was held by the capacitor. The integration started when the isolation gate was shut off. At the end of the integration time, both row selection transistors were selected, and the differential output was read by the column readout circuit. Since the reset noise voltage was held by the capacitor, it was eliminated from the differential output. Noise due to dark current of the photodiode and diffusion capacitor could be characterized in dark and subtracted from the output. While row selection transistors were still on, the pixel was reset by turning on both reset transistor and isolation gate. The differential output was read out again and subtracted from the one read before reset. Thus offset of the pixel could be further removed.

2. Results

The differential pixel shown in FIG. 7 was fabricated using a commercial 0.5 μm CMOS process. The reset noise was tested and compared with that obtained from a three transistor one diode active pixel sensor, which is often used in consumer CMOS digital cameras. The results are presented in Table 8. The reset noise of the differential pixel in both single and differential operation modes was tested under illumination and in darkness, respectively. The reset noise of the conventional APS was also tested under the same illumination conditions. As shown in Table 7, the differential pixel has the same noise voltage as normal APS when working in single-ended readout mode. When operated differentially, the reset noise was reduced by a factor of 10 under illumination and by a factor of almost 32 in darkness.

TABLE 7
Fluorescence sensor results

| Sensors | In darkness (V) | Under illumination (V) |
|---|---|---|
| Photodiode_SH | Mean = 1.665164; | Mean = 1.665226; |
| (single-ended readout) | var = 0.0024553 | var = 0.0023242 |
| Photodiode_SH | Mean = 4.592966e−005; | Mean = 0.01103126; |
| (-differential readout) | var = 7.5824e−005 | var = 0.0002055 |
| Photodiode_APS | Mean = 3.386899; | Mean = 3.38634; |
| (CDS readout) | var = 0.0020626 | var = 0.0020806 |

### Example 3 CMOS Capacitance Sensor for Cell Proximity Detection

Capacitance measurements are commonly used for applications such as fingerprint sensing, position sensing and interconnect characterization. In this work, the technique was adapted to cell proximity detection for evaluating the surface adhesion properties of living cells in cell clinics.

1. Design

A custom CMOS capacitance sensor for cell proximity detection has been designed using the topology shown in FIG. 6 (Lee et al., 1999). The physical principle underlying operation of the sensor is charge sharing. The coupling capacitance C_{cell} is formed by the series combination of the capacitances between the cell and the passivation layer and between the passivation layer and the topmost metal electrode. C_{cell} varies-with the strength of coupling of the cell to the chip surface.

The sensor circuit had two nodes, N1 and N2, with parasitic capacitances C_{N1} and C_{N2}. Charging and discharging of these nodes were controlled by a set of three MOSFET switches: M1, M2 and M3, in two phases of operation. In the reset phase, switches M1 and M3 were turned on, charging N1 to Vdd and N2 to Vss, while switch M2 was off. In the evaluation phase, M2 was turned on. The joint nodal voltage V_{N} as a result of the charge redistribution can be expressed as:

$${V}_{N} = \frac{( {C}_{N 1} + {C}_{cell} ) Vdd + {C}_{N 2} Vss}{{C}_{N 1} + {C}_{N 2} + {C}_{cell}} \qquad ( 2 )$$

where C_{cell} is the capacitance being sensed. As C_{cell} increases with increasing cell proximity to the surface, so does VN. This determines the capacitance to voltage mapping. In order to maximize the sensitivity of the circuit, the parasitic nodal capacitances must be minimized. The sensor dynamic range also increases with increasing area of the metal electrode plate.

In this embodiment (for an example, see FIG. 6), the topmost metal layer (in this case metal3), formed the sensing electrode. The fringe capacitances between the metal3 plate and the substrate were shielded by means of a larger area metal2 plate below the sensing electrode. The large capacitance between metal2 and metal3 plates was cancelled by driving the metal2 shield with a potential that tracks the sensing electrode potential using a unity-gain buffer.

The sensor was designed for a supply voltage of +/−1.5 V and was fabricated in a commercially available, 0.5 μm CMOS technology with three metal layers. Three sensors with electrode areas of 20×20 μm², 30×30 μm² and 40×40 μm² were designed and tested.

2. Calibration

In order to translate the sensor outputs to sensed capacitance values, the output voltages during the evaluation phase were subtracted from their corresponding reset voltages for offset cancellation. It follows from (4) that the sensed capacitance depends on this voltage difference according to the expression improves with increasing proximity to the surface, increasing electrode area and decreasing noise level. The sensors exhibited a distance resolution of under 3 nm when the sensed object was in close proximity to the chip surface.

3. Results

a. Sensor Testing Using an External Metal Electrode

The transducer was calibrated by using an external metal electrode which vertical positioning was controlled by means of a piezoelectric micropositioner. FIG. 8 shows the test results superimposed on the simulated sensor voltages. The symbols represent experimental values of sensor voltage obtained by moving the micropositioned electrode in steps of 2 to 3 μm. The simulation parameters were: passivation layer thickness of 1 μm; a dielectric constant of 6; CN1=20 and CN2=18 fF. The output voltage dynamic ranges for the 20×20 (x's), 30×30 (open circles) and 40×40 (+'s) sensors were found to be 100 mV, 200 mV and 400 mV respectively.

b. Sensor Resolution Analysis

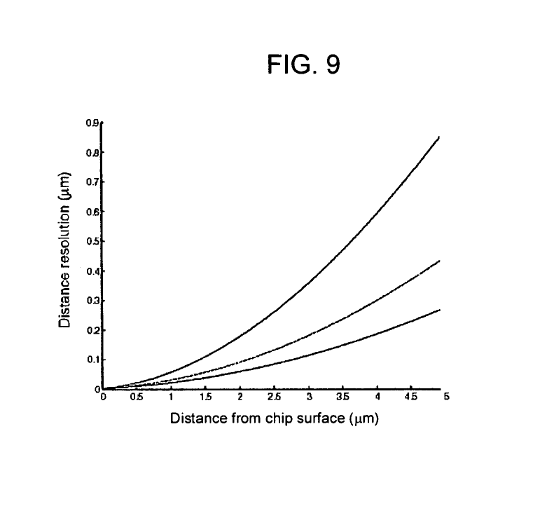

Sensitivity was a function of proximity to chip surface, with the sensor being highly sensitive to distance when the cell was closer to the surface. This characteristic was appropriate for the present application, since the cells were directly coupled to the chip surface. Capacitance resolution depended upon the noise performance of the circuit and the test setup. Simulation of the sensor circuit gives an output noise level of 1 mV, which corresponds to an expected capacitance resolution of 30 aF. Noise in the actual experimental setup was measured to be 5 mV, which corresponds to a capacitance resolution of 135 aF. FIG. 9 shows these results, where the 20×20 sensor is represented by a dotted line; 30×30 by a dashed line, and 40×40 by a solid line.

c. Sensor Response to Living Cells

The sensor chip in a 40 pin dual in-line (DIP) ceramic package was encapsulated using a biocompatible material in order to insulate the bonding wires and to isolate the cells from toxic materials in the chip package. A well was formed on top of the chip surface for containing the cells in growth medium. The cell culture medium was itself an ionic solution and forms a conducting layer above the surface. The sensor chip was first calibrated by adding the medium alone without cells and measuring the capacitive coupling between the solution and electrodes. The well was then loaded with bovine aortic SMC's, and the sensor outputs were monitored over 24 hours. In between measurements, the fixture was maintained in an incubator at 37° C., 5% CO₂. The fixture was loaded with a very concentrated solution of cells, so all sensors in the test array were exposed to similar conditions. Coupling of cells to every sensor was confirmed through visual observation.

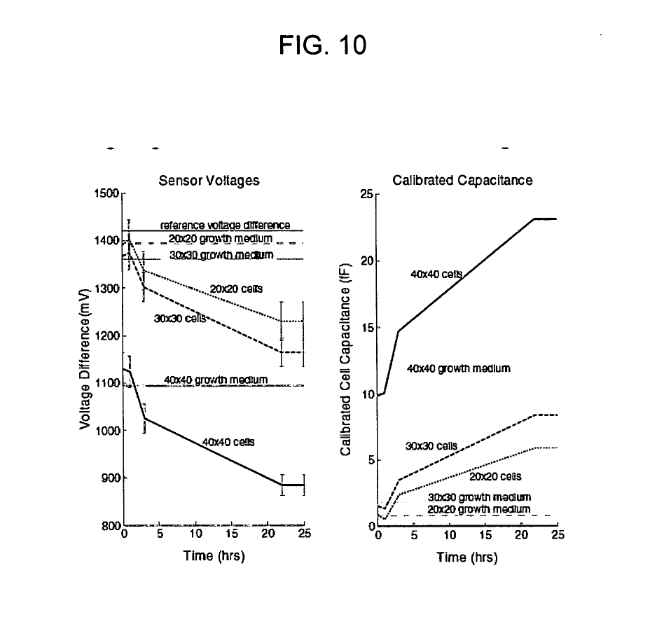

The sensed capacitances were calibrated as discussed above. FIG. 10 shows a plot of the average voltage differences for the three sensors, with all values aligned according to the zero sensed capacitance reference. Error bars indicate the standard deviation in response between all sensors of the same size. The voltage differences for all three sensors decreased with time, tracking the adhesion process as expected and indicating an increase in capacitive coupling between the cells and the on-chip electrodes after they were allowed to settle on the chip surface over a period of time. The sensor output voltages changed by an average of 125 mV, 150 mV and 175 mV for the 20×20, 30×30 and 40×40 sensors respectively, over the 24 hour period. Based upon these measurements the calibrated cell capacitances varied from sub-fF to around 10-20 fF, with different sensing ranges for the three sensors as shown in FIG. 10.

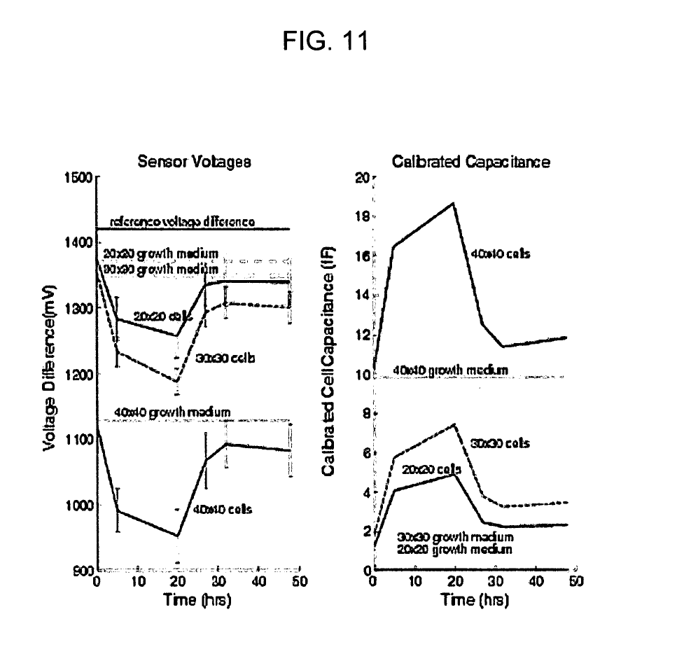

A second experiment with the SMC's monitored the sensor response to changes in cell viability. For this, the above test procedure was repeated, but this time with SMC's stained with neutral red in a colorless growth medium. The sensors were monitored over a period of 48 hours. Viability was assessed independently through visual inspection of the stained cells. Living healthy cells have the characteristic property of taking up and retaining neutral red stain whereas non-viable cells do not retain the stain. FIG. 11 shows the sensor response over the 48 hour period. Over the first day, the cells were able to retain the stain and the sensors showed an increase in capacitive coupling between cells and sensor electrodes. On the second day, however, it was observed that the cells no longer retained the stain and had released the dye into the growth medium, an indication of non-viability. Accordingly the sensors showed a decrement in the measured capacitance values.

### Example 4 CMOS Contact Imager

Conventional digital imaging is a mature technology commonly used to acquire images of cells. Typically these images are acquired using a light microscope, wherein optical elements such as lenses focus the image onto an imaging array (either the eyes of a human observer or an electronic sensor). In this work, a “contact” imaging configuration has been developed which does not use intervening optics and which generates a representation of a specimen directly coupled to the surface of the chip.

An embedded optical image sensor, called a contact imager, for imaging of a biological specimen directly coupled to the chip surface was fabricated and tested. The designed CMOS image sensor comprised an array of active pixel sensors (APS), logic and control signal generation, and readout circuits. The pixel layout had a pitch of 8.4 μm (24λ). The design was fabricated in a commercially available 0.5 μm CMOS technology. The imager was first characterized as a normal CMOS image sensor, and then as a contact imager with microbeads (16 μm) placed directly on the chip surface. After further packaging with bio-compatible material, the chip was tested with cells cultured directly on the chip surface. Test results confirm successful detection of both beads and cells.

Major characteristics of the fabricated chip are summarized in Table 8.

TABLE 8
Major characteristics of fabricated chip

| Process | AMI05 (SCMOS design rule, λ = 0.35 um) | |
|---|---|---|
| Power supply | 5 | V |
| Maximum signal | 1.2 | V |
| Conversion gain | 22 | uV/e |
| Meas. pixel noise | σ = 2.5 mV over 2 ms | |
| Dynamic range | 53.6 | dB |
| Dark signal | 0.46 | V/sec |

1. Design and Operation
a. Pixel Design

A schematic for the CMOS photodiode type APS is shown in FIG. 6. Several techniques were used in order to achieve a small pixel size. First, all three MOS transistors were N-type transistors. An Nplus-Psub photodiode was used to avoid minimum Nwell spacing requirements. To reduce the number of contacts, there was only one Vdd contact per pixel. The layout of the pixel array is preferably staggered so that one Vdd contact can be shared by the source follower input transistor of one pixel and the reset transistor of another. The reset signal is routed through rows using only Poly1. Thus, a small pixel size with maximum optically active area can be achieved. We used the MOSIS scalable CMOS (SCMOS) design rules for a double poly, three metal layer, Nwell process (λ=0.35 μm). A pitch size of 8.4 μm (24λ) was achieved. The topmost metal layer (metal3) was used for routing Vss and blocks light from all but the photodiode active area. The fill factor, calculated as the ratio of uncovered photodiode active area to the total pixel area, was 17%.

b. Contact Imager Architecture and Operation

The system consisted of a 96×96 APS array, row and column scanners, column-wise readout circuits, and buffers and switches for input control and clock signals. Scanners and readout circuitry were implemented according to known art. The row and column scanner was implemented using a closed-loop shift register where each stage was a positive-edge triggered dynamic D-flip-flop. The output of the first stage of the row scanner served as the clock signal for the column scanner. The complete chip including the pad frame fit on a standard 1.5 mm×1.5 mm die.

A schematic diagram of one pixel together with circuits for row logic and control, and correlated double sampling (CDS) readout chain is shown in FIG. 5A along with a timing diagram (FIG. 5B). Three clocks were required to operate the imager: ph_1, ph_2, and ph_clamp. They shared the same frequency and satisfied the phase relationships indicated by the dashed lines in FIG. 5B. The clock signal for the row scanner was ph_1. The output of one stage of the row scanner served as the Rowselect signal for all pixels in the corresponding row. The Reset signal initialized the integrated pixel value and was generated by performing a logic AND operation on the signals ph_2 and Row_select.

To suppress 1/f noise and fixed pattern noise (FPN) due to threshold variations of source-follower input transistors, column-wise CDS was performed (White et al., 1974). After the pixel was selected by Row_select and before Reset goes high, clock ph_clamp was high. At this point the integrated voltage signal Vₒᵤₜ(t1) was read out from the column amplifier. Clock ph_clamp then became low right before the positive edge of the Reset signal. This turned the input of the readout amplifier into a floating node capacitively coupled to the output of the selected pixel. After Reset goes high, the voltage signal Vₒᵤₜ(t2) was sampled again, where V_{signal}=Vₒᵤₜ(t2)−Vₒᵤₜ(t1) was the difference of pixel outputs before and after the photodiode was reset as shown in FIG. 5A. In order to perform CDS properly, the three clock signals must satisfy the following phase shifts: clock ph_1 is an inverted and slightly delayed copy of clock ph_2 so that pixels are not reset right after they are selected. Clock ph-clamp is an inverted and slightly advanced version of ph_2. It was especially important that the rising edge of ph_2 fall behind the falling edge of ph_clamp. Otherwise, V_{signal} would not have been coupled to the column output. In order to illustrate these phase shifts clearly, the clock signals shown in FIG. 5B are not shown to scale.

c. Experimental Results

First, the chip was aligned with a camera objective and its operation as an imager was verified. Dummy pixels surrounding the pixel array minimize edge effects within the pixel array that cause dark pixels along the edges. The chip was then tested as a contact imager using microbeads placed directly on the chip surface. After being further packaged with bio-compatible material, the chip was tested with cells plated on chip surface.

The contact imager was first tested on the bench. Three clocks of frequency 50 kHz, with phase shifts as described above were generated from a microcontroller. Another clock of frequency 100 kHz was also generated to provide timing signals for a PC-hosted data acquisition card (DAQ) (MCC PCI-DAS6052). Synchronization was achieved by triggering both the on-chip scanner and the data acquisition using a pulse signal generated by the DAQ card.

The contact imager was tested with dry, 16 μm diameter polymer microspheres placed directly on the chip surface. The contact imager was capable of detecting-cell-size particles in a precise manner.

To test the imager chip with cells, the chip was packaged in a standard 40 pin ceramic dual in-line package (DIP). In order to test the contact imager with cultured cells directly coupled to the chip surface, the chip was further packaged both to protect the bond pads and wires from being corroded and shorted by cell culture medium and to protect cells from toxic materials used in the chip packaging. A photo-patternable polymer (Loctite® 3340; available from R.S. Hughes Company, Inc.; Sunnyvale, Calif.) was used to encapsulate all bonding pads and wires and to leave an opening about 1 mm×1 mm large in the center of the die. These experiments determined that Loctite® 33340 was suitable for packaging the die for more than a week.

On top of the Loctite® packaged chip, a piece of plastic tube was glued to form a well. The well was sufficiently large to contain enough cell culture medium to prevent the cells on the chip surface from drying out. SMCs (Cell Applications, Inc.) were stained using neutral red dye to increase their visibility. The cells were easily monitored, and their positions corresponded perfectly with the positions shown in a photomicrograph of the chip surface.

### Example 5 Protein-Conjugated QDs for Sensing Protein Dynamics in Living Cells

1. Monitoring Protein-Protein Interactions

Quantum dots (QDs) can be used for monitoring protein-protein interaction as following. First, a cell is engineered to express a fluorescent protein-tagged protein (i.e., a protein with a fluorescent protein attached to it). Second, a recombinant protein or protein fragment is tethered to a quantum dot. The protein-QD complex is transferred to the engineered cell using conventional methods, such as microinjection or using liposomes. The fluorescent protein-tagged protein interacts with the protein tethered to the QD when the signaling pathway of which these two proteins are members is activated following pathogen contact with the cell.

The interaction between the protein on the QD and the fluorescent protein-tagged protein is detected by FRET. FRET involves a donor and an acceptor. The donor is excited at one wavelength, the excitation wavelength, and if the acceptor is in close proximity to the donor, enough energy can be transferred to the acceptor that the acceptor can radiate at a second, emission, wavelength. The emissision can be recorded by a detector or camera. Practically, the QD is typically the donor because it can be excited over a wide spectrum of frequencies, it emits over a narrow range of frequencies, and it can be excited over long periods of time before significant photo-bleaching. The fluorescent protein-tagged protein is the acceptor.

A QD-fluorescent protein pair for which FRET occurs is a green CdSe-ZnS core QD (wavelength of 555 nm) and DsRed protein. Because the emission spectrum of 555-nm QDs overlaps with the excitation spectrum of the DsRed protein, enough energy transfer can occur when the QD and the DsRed protein are in close proximity (nanometer range), e.g., when the proteins to which they are attached are interacting.

Using conventional molecular biology methods, it is important to design the DsRed protein tag so that it does not interfere with the binding capability of the protein to which DsRed protein is attached. If the binding domain of that protein is in the C-terminal domain, then Dsred should be attached to the N-terminal domain.

Design of this protein complex can be also aided by computational methods using the known crystallographic structures of the protein and the fluorescent protein. Instead of using a fluorescent protein (e.g. DsRed), one could use a fluorescent dye. In this particular application, an example of dye is rhodamine red.

Quantum dots conjugated with functional proteins to monitor in live cells protein dynamics and protein-protein interactions in real time were designed and tested. In this example, results are presented when negatively charged dihydroxylipoic acid (DHLA)-coated (capped) quantum dots were conjugated with the protein EB1 through a His-tag at its C-terminus (e.g., such as the amino acid sequence SEQ ID NO:1 (GenBank Accession No. Q15691), shown in Table 9). EB1 is a protein complex that binds the fast growing ends of microtubules (Schuyler and Pellman, 2001). In this example, EB1 was used as a marker of microtubule dynamics in live cells.

TABLE 9
Microtubule-associated protein RP/EB family member 1 (APC-
binding protein EB1) from Homo sapiens (SEQ ID NO:9)

```
Met Ala Val Asn Val Tyr Ser Thr Ser Val Thr Ser Asp Asn Leu Ser
1 5 10 15
Arg His Asp Met Leu Ala Trp lle Asn Glu Ser Leu Gln Leu Asn Leu
                        20 25 30
Thr Lys Ile Glu Gln Leu Cys Ser Gly Ala Ala Tyr Cys Gln Phe Met
                35 40 45
Asp Met Leu Phe Pro Gly Ser Ile Ala Leu Lys Lys Val Lys Phe Gln
        50 55 60
Ala Lys Leu Glu His Glu Tyr Ile Gln Asn Phe Lys Ile Leu Gln Ala
65 70 75 80
Gly Phe Lys Arg Met Gly Val Asp Lys Ile Ile Pro Val Asp Lys Leu
                                85 90 95
Val Lys Gly Lys Phe Gln Asp Asn Phe Glu Phe Val Gln Trp Phe Lys
                        100 105 110
Lys Phe Phe Asp Ala Asn Tyr Asp Gly Lys Asp Tyr Asp Pro Val Ala
                115 120 125
Ala Arg Gln Gly Gln Glu Thr Ala Val Ala Pro Ser Leu Val Ala Pro
        130 135 140
Ala Leu Asn Lys Pro Lys Lys Pro Leu Thr Ser Ser Ser Ala Ala Pro
145 150 155 160
Gln Arg Pro Ile Ser Thr Gln Arg Thr Ala Ala Ala Pro Lys Ala Gly
                                165 170 175
Pro Gly Val Val Arg Lys Asn Pro Gly Val Gly Asn Gly Asp Asp Glu
                        180 185 190
Ala Ala Glu Leu Met Gln Gln Val Asn Val Leu Lys Leu Thr Val Glu
                195 200 205
Asp Leu Glu Lys Glu Arg Asp Phe Tyr Phe Gly Lys Leu Arg Asn Ile
        210 215 220
Glu Leu Ile Cys Gln Glu Asn Glu Gly Glu Asn Asp Pro Val Leu Gln
225 230 235 240
Arg Ile Val Asp Ile Leu Tyr Ala Thr Asp Glu Gly Phe Val Ile Pro
                                245 250 255
Asp Glu Gly Gly Pro Gln Glu Glu Gln Glu Glu Tyr
                        260 265
```

2. Results

QDs were mictoinjected into living cells (Swiss 3T3 fibroblasts; ATCC Deposits CCL-163 and CCL-92 (Todaro and Green, 1963)), which were then tested for viability at different times post-injection. Microinjection circumvented the endocytotic pathway and subsequent directed motion paused by passive QD engulfment by the cell or cell targeting using, for example, lipfection agents. Microinjection was successful, and the cells recovered from the trauma of injection as rapidly as for mock injection of injection buffer. However, DHLA-capped QDs aggregated within ˜60 minutes; aggregation also occurred. To address this issue, the observation that DHLA QDs coated with maltose binding protein (MBP) did not aggregate in the cytoplasm of Swiss 3T3 fibroblasts for at least 24 h post-injection was exploited. This success could have been due to the fact that MBP is not naturally expressed in mammalian cells. Therefore, we purified His-tag human EB1 proteins and allowed them to bind DHLA-capped QDs and tested if B1 had the same stabilizing effect as MBP. QDs conjugated with EB1 abrogated aggregation of DHLA QDs for at least 24 hours. Moreover, as in the case of unconjugated DHLA-capped QDs, mock microinjection and microinjection with EB1 QD bioconjugates caused neither significant morphological changes of the cell nor cell death, as tested by trypan blue assay. Therefore, the functionalization of the DHLA QDs with EB1 had the important benefit of stabilizing the QDs while leaving the cell mostly intact.

### ABBREVIATIONS

TABLE A

| Abbreviation | Definition |
|---|---|
| Ag | Silver |
| AgCl | Silver chloride |
| ATCC | American Type |
| | Culture Collection |
| BCB | Benzocyclobutene |
| CMOS | Complementary |
| | metal oxide silicon |
| DBS | Dodecylbenzene |
| | sulfonate |
| DNA | Deoxyribonucleic |
| | acid |
| GFP | Green fluorescent |
| | protein |
| IAP | Inhibitor of |
| | apoptosis |
| LED | Light-emitting diode |
| MOSFET | Metal-oxide- |
| | semiconductor field- |
| | effect transistor |
| NaDBS | Sodium |
| | dodecylbenzene |
| | sulfonate |
| PIN | Positive-intrinsic- |
| | negative |
| PPy | Polypyrrole |
| QD | Quantum dot |
| RF | Radio frequency |
| Si | Silicon |
| SiO₂ | Silicon dioxide |
| VCSEL | Vertical-cavity |
| | surface-emitting laser |
| VLSI | Very large scale |
| | integration |

### REFERENCES

Alberts, B., A. Johnson, J. Lewis, M. Raff, K. Roberts, and P. Walter. 2002. Programmed cell death (Apoptosis). In Molecular Biology of the Cell. Garland Publishing, Inc., New York, N.Y. 1010-1014.
Asian, K., J. R. Lakowicz, H. Szmacinski, and C. D. Geddes. 2004. Metal-enhanced fluorescence solution-based sensing platform. J. Fluoresc. 14:67-9.
Clark, D., G. Cypret, C. Gonsalves, N. Hoesly, A. Moore, N. Saulnier, P. Scales-Brown, M. Slack, and R. White. 2001. Biological detection systems technologies: Technology and industrial base study. TRW Systems and Information Technology Group.
Culurciello, E., and A. Andreou. 2004. 16×16 pixel silicon on sapphire CMOS digital pixel photosensor array. Electronics Letters. 40:66-68.
Culurciello, E., R. Etienne-Cummings, and K. Boahan. 2001. Arbitrated address-event representation digital image sensor. Electronics Letters. 37:1443-1445.
Doty, R. C., D. G. Fernig, and R. Levy. 2004. Nanoscale science: a big step towards the Holy Grail of single molecule biochemistry and molecular biology. Cell Mol Life Sci. 61:1843-9.
Herman, B. 1998. Fluorescence microscopy. Springer, N.Y.
Jager, E. W., E. Smela, and O. Inganas. 2000. Microfabricating conjugated polymer actuators. Science. 290:1540-5.
Johnson-Winegar, A. 2000. The DoD biological detection program NDIA roundtable discussions. Department of Defense.
Kerr, J. F., A. H. Wyllie, and A. R. Currie. 1972. Apoptosis: a basic biological phenomenon with wide-ranging implications in tissue kinetics. Br J Cancer. 26:239-57.
Lee, J.-W., D.-J. Min, J. Kim, and W. Kim. 1999. 600-dpi Capacitive fingerprint sensor chip and image-synthesis technique. IEEE JSSC. 34:469-475.
Lidke, D. S., and D J. Arndt-Jovin. 2004. Imaging takes a quantum leap. Physiology (Bethesda). 19:322-5.
Roulet, J.-C., R. Völkel, H. Herzig, E. Verpoorte, N. d. Rooij, and R. Dändliker. 2001. Fabrication of multilayer systems combining microfluidic and microoptical elements for fluorescence detection. J. Microelectromech. Syst. 10:482-491.
Schuyler, S. C., and D. Pellman. 2001. Microtubule “plus-end-tracking proteins”: The end is just the beginning. Cell. 105:421-4.
Skotheim, T. A., R. L. Elsenbaumer, and J. R. Reynolds. 1998. Handbook of conducting polymers. M. Dekker, New York. xiii, 1097 p. pp.
Smela, E. 1999. Microfabrication of PPy microactuators and other conjugated polymer devices. J. Micromechanics and microengineering. 9:1-18.
Smith, A. M., and S. Nie. 2004. Chemical analysis and cellular imaging with quantum dots. Analyst 129:672-7.
Todaro, G J., and H. Green. 1963. Quantitative studies of the growth of mouse embryo cells in culture and their development into established lines. J Cell Biol. 17:299-313.
Weinrauch, Y., and A. Zychlinsky. 1999. The induction of apoptosis by bacterial pathogens. Annu Rev Mirobiol. 53:155-87.
White, M., D. Lampe, F. Blaha, and I. Mack. 1974. Characterization of surface channel CCD image arrays at low light levels. IEE JSSC. 9:1-13.
Wyllie, A. H., J. F. Kerr, and A. R. Currie. 1980. Cell death: the significance of apoptosis. Int Rev Cytol 68:251-306.

We claim:

1. A device for detecting at least one pathogen, comprising:
at least one cell that produces a signal upon contact with the pathogen;
at least one cell clinic on a surface of a chip for containing the cell; and
an on-chip means for detecting the signal,
wherein the cell specifically produces a signal when exposed to the pathogen, and detecting the signal correlates with the presence of the pathogen.

2. The device of claim 1, wherein detecting the signal comprises detecting a plurality of signals from at least one cell.

3. The device of claim 2, wherein the cell specifically produces a signal when exposed to a first and second pathogen, wherein the first pathogen elicits a specific first signal, and the second pathogen elicits a second specific signal.

4. The device of claim 1, wherein the cell is a modified cell.

5. The device of claim 1, wherein the signal comprises fluorescence or fluorescence resonance energy transfer.

6. The device of claim 1, wherein the signal comprises light emission or cessation of light emission.

7. The device of claim 1, wherein the signal comprises at least one change, the change selected from the group consisting of cell resistance, cell impedance, cell capacitance, cell ion concentration, cell or medium pH, carbon dioxide concentration, nutrient concentration, cell waste concentration, cellular mechanical properties, cell position, cell number, and temperature.

8. The device of claim 4, wherein the cell modification comprises an exogenous marker, an exogenous polynucleotide, or a virus.

9. The device of claim 8, wherein the marker comprises a polypeptide, a nanoparticle, a quantum dot, or a dye.

10. The device of claim 1, wherein the means for detecting a response comprises at least one sensor.

11. The device of claim 10, wherein the at least one sensor comprises a sensor for fluorescence, carbon dioxide, pH, ion concentration, cell resistance, cell impedance, cell capacitance, cell waste, cellular mechanical properties, cell position, cell number nutrient or temperature.

12. The device of claim 11, wherein the sensor comprises a fluorescence sensor.

13. The device of claim 12, wherein the fluorescence sensor comprising a current mode pixel, an APS pixel, or a single-photon avalanche detector.

14. The device of claim 13, wherein the sensor further comprises an optical filter.

15. The device of claim 14, wherein the light filter is a notch filter, a band-pass filter, or a low-pass optical filter.

16. The device of claim 14 wherein the optical filter comprises an absorbing dye, an interference filter, a distributed Bragg reflector, or a patterned light-blocking layer.

17. The device of claim 1, wherein the chip comprises integrated circuitry.

18. The device of claim 17, wherein the integrated circuitry comprises complementary metal oxide semiconductor technology.

19. The device of claim 1, wherein the cell clinic further comprises an actuated lid.

20. The device of claim 19, wherein the actuator of the lid comprises polypyrrole.

21. The device of claim 19, wherein the lid comprises a semi-permeable membrane.

22. The device of claim 1, further comprising a light source that directs light to at least one cell in the cell clinic.

23. A device for detecting pathogens, comprising:
a plurality of cells-that produce at least one signal upon contact with a pathogen;
a cell clinic comprising a plurality of vials on a surface of a chip for containing the cells, each vial containing at least one cell responsive to a specific pathogen; and
a means for detecting responses of the cells to the pathogens,
wherein the cells specifically produce at least one signal to the pathogens, and detecting the signal correlates with the presence of at least one pathogen.

24. The device of claim 23, wherein at least one cell specifically responds to a first and second pathogen, wherein the first pathogen elicits a specific first response, and the second pathogen elicits a second specific response.

25. The device of claim 23, wherein n and m represent integers greater than or equal to 1, and
m cells specifically respond to n stimuli, wherein an n^{th} stimulus elicits a specific response in an m^{th} cell.

26. The device of claim 23, wherein the pathogen elicits a sequence of signals from at least one cell over time.

27. The device of claim 26, wherein detecting signal cell comprises detecting the sequence of responses.

28. The device of claim 23 further comprising an array of sensors.

29. The device of claim 23, wherein the plurality of vials contain different types of cells.

30. A method of analyzing a sample, comprising:
introducing the sample into a cell canary, the cell canary comprising a cell that produces a specific signal in response to a pathogen; and
assaying the cell for the signal, wherein detecting the signal correlates with the presence of the pathogen.

31. The method of claim 30, wherein the signal comprises fluorescence.

31. A method of making a device for detecting a pathogen, comprising:
fabricating on a chip at least one means for detecting a signal from at least one cell having a specific signal in response to a pathogen;
fabricating at least one cell clinic on a surface of the chip for containing the cell; and
loading the cell into the cell clinic.
